SEQUENCE LISTING (13 sequences)

SEQ ID NO: 1 - protein, 214 aa, Artificial Sequence
  Complete light chain sequence (LC4) of humanised LC4HC3
  MISC_FEATURE (1)..(214)
EIVMTQSPDSLAVSLGERATINCKASQNVDTYVVWYQQKPGQPPQPLIYSASSRFSGVPDRFSGSGSGTDFTLTISSLQAEDVAIYYCQQYHNSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 234 aa, Artificial Sequence
  Complete light chain sequence (LC4) of humanised LC4HC3 in combination with signal peptide used in CHO cells expression system; Signal peptide underlined
  MISC_FEATURE (1)..(234)
MVSSAQFLGLLLLCFQGTRCEIVMTQSPDSLAVSLGERATINCKASQNVDTYVVWYQQKPGQPPQPLIYSASSRFSGVPDRFSGSGSGTDFTLTISSLQAEDVAIYYCQQYHNSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 107 aa, Artificial Sequence
  Light chain (LC4) variable region of humanised LC4HC3
  MISC_FEATURE (1)..(107)
EIVMTQSPDSLAVSLGERATINCKASQNVDTYVVWYQQKPGQPPQPLIYSASSRFSGVPDRFSGSGSGTDFTLTISSLQAEDVAIYYCQQYHNSPLTFGGGTKVEIK

SEQ ID NO: 4 - protein, 449 aa, Artificial Sequence
  Complete heavy chain sequence (HC3) of humanised LC4HC3
  MISC_FEATURE (1)..(449)
QVQLVQSGAEVKKPGASVKVSCKASGYIFIDYGMHWVRQAPGQRLEWMGSINTKSGVSTYAAEFKGRVTIYSDTSASTAYMELSSLRSEDTAVYFCARPPYYSQYGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 468 aa, Artificial Sequence
  Complete heavy chain sequence (HC3) of humanised LC4HC3 in combination with signal peptide used in CHO cell expression system; Signal peptide underlined
  MISC_FEATURE (1)..(468)
MGWTLVFLFLLSVTAGVHSQVQLVQSGAEVKKPGASVKVSCKASGYIFIDYGMHWVRQAPGQRLEWMGSINTKSGVSTYAAEFKGRVTIYSDTSASTAYMELSSLRSEDTAVYFCARPPYYSQYGSYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 119 aa, Artificial Sequence
  Heavy chain (HC3) variable region of humanised LC4HC3
  MISC_FEATURE (1)..(119)
QVQLVQSGAEVKKPGASVKVSCKASGYIFIDYGMHWVRQAPGQRLEWMGSINTKSGVSTYAAEFKGRVTIYSDTSASTAYMELSSLRSEDTAVYFCARPPYYSQYGSYWGQGTLVTVSS

SEQ ID NO: 7 - protein, 214 aa, Artificial Sequence
  Complete light chain sequence (LC0) of chimeric LC0HC0
  MISC_FEATURE (1)..(214)
DIVMTQSQKFMSTSVGDRVSVTCKASQNVDTYVVWYQQKPGQSPKPLIYSASSRFSGVPDRFTGTGSGTDFTLTINNVQSEDLAEYFCQQYHNSPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 107 aa, Artificial Sequence
  Light chain (LC0) variable region of chimeric LC0HC0
  MISC_FEATURE (1)..(107)
DIVMTQSQKFMSTSVGDRVSVTCKASQNVDTYVVWYQQKPGQSPKPLIYSASSRFSGVPDRFTGTGSGTDFTLTINNVQSEDLAEYFCQQYHNSPLTFGGGTKLEIK

SEQ ID NO: 9 - protein, 449 aa, Artificial Sequence
  Complete heavy chain sequence (HC0) of chimeric LC0HC0
  MISC_FEATURE (1)..(449)
QVHLVQSGPELKKPGETVKISCKASGYIFIDYGMHWVKQAPGKGLKWMGSINTKSGVSTYAAEFKGRFAFSLETSASTAYLQINNLKNEDTATYFCARPPYYSQYGSYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 119 aa, Artificial Sequence
  Heavy chain (HC0) variable region of chimeric LC0HC0
  MISC_FEATURE (1)..(119)
QVHLVQSGPELKKPGETVKISCKASGYIFIDYGMHWVKQAPGKGLKWMGSINTKSGVSTYAAEFKGRFAFSLETSASTAYLQINNLKNEDTATYFCARPPYYSQYGSYWGQGTLVTVSA

SEQ ID NO: 11 - DNA, 705 bp, Artificial Sequence
  Polynucleotide encoding LC4 incl. signal peptide
  misc_feature (1)..(705)
atggtcagctctgctcaatttctcggactccttcttctgtgctttcaaggaacacgctgcgagatcgtgatgactcagtccccggactcactggcagtgtccttgggcgaaagagccaccatcaactgtaaagccagccagaacgtggacacctacgtggtctggtaccagcagaagcctggacagccaccgcagccgttgatctactcggcctcatcaaggttctccggggtgccggaccgcttctccggatccggctccggcaccgatttcaccctgaccatctcctcactgcaagccgaggacgtggctatctactattgccagcagtaccacaactccccactgaccttcggtggcggaactaaggtcgagattaagcggaccgtggcggccccctctgtgttcattttccctccctcggacgaacagctgaagtcgggaacagcctccgtcgtgtgcctgctcaacaacttctacccccgggaagcgaaggtccagtggaaagtggataacgcactccaatcggggaactcccaggaatccgtgactgagcaggactcgaaggattccacttactccctgtcgtccaccctgactctgagcaaggccgactacgagaagcataaggtctacgcctgcgaagtgacccaccagggtctgagctcccctgtgaccaagagctttaatcggggcgaatgttga

SEQ ID NO: 12 - DNA, 1407 bp, Artificial Sequence
  Polynucleotide encoding HC3 incl. signal peptide
  misc_feature (1)..(1407)
atgggttggaccctcgtctttctgttccttctttccgtcaccgctggagtgcatagccaggtccaattggtgcagtcaggcgccgaagtgaaaaagcctggggcgtcggtgaaagtgtcctgcaaagcctcgggctacatctttattgactacggaatgcactgggtccgccaggccccgggccagaggctggagtggatgggatccattaacaccaagagcggagtgtcaacttacgcagccgagttcaagggacgggtgaccatctatagcgatacctctgcgtcgaccgcctacatggaattgtcatcactccggtccgaggacactgccgtgtacttctgcgcaaggccaccctactactcgcaatacggcagctactggggccagggaacacttgtgaccgtgtcgagcgcgtccaccaagggtccctccgtgttccctctcgcgccgtcctcaaagtctacctccggtggaactgccgcgctcggttgtctcgtgaaggactacttcccggagcctgtgactgtctcctggaactccggggccctcaccagcggagtgcacactttccccgccgtgctgcaatcctccggcctgtacagcctgtcctccgtcgtgactgtgcctagctcctccctgggaacccagacctacatctgcaacgtgaaccacaagccctccaacaccaaggtcgacaagaaggtcgaaccgaagtcgtgcgacaagactcatacgtgccctccttgcccggccccggaactgctgggaggcccatccgtgttcctgttcccacccaagcctaaggataccctgatgatcagcagaacaccggaagtgacctgtgtggtggtggacgtcagccacgaagatcccgaggtcaagttcaattggtacgtggacggggtggaggtgcacaacgcaaagaccaagccccgggaggaacagtacaactccacctatcgcgtggtgtcggtgctgacggtgctgcaccaggactggttgaacggaaaggagtataagtgcaaagtgtcgaacaaggccctgcccgctcctatcgaaaagaccatctccaaggccaagggccagccgcgggaaccccaggtctacactctcccaccgagccgcgacgaactgactaagaatcaagtgtcgctgacttgcctcgtcaagggcttctacccgtccgacatcgccgtggaatgggagagcaacggccagccggaaaacaactacaagaccacccctcccgtgctggattccgacgggtccttcttcctgtactcaaaactgaccgtggataagtccagatggcagcagggcaatgtcttttcatgctccgtgatgcacgaggctctgcataaccactacacccagaagtcgctgtccctgtccccggggaagtga

SEQ ID NO: 13 - protein, 234 aa, Artificial Sequence
  Complete light chain sequence (LC3) of humanised LC3HC3
  MISC_FEATURE (1)..(234)
MVSSAQFLGLLLLCFQGTRCDVVMTQSPDSLAVSLGERVTINCKASQNVDTYVVWYQQKPGQSPKLLIYSASSRFSGVPDRFSGSGSGTDFTLTISSLQAEDVAVYYCQQYHNSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0302977 A1
(43) Pub. Date: Oct. 2, 2025

(54) ANTIBODY-DRUG CONJUGATES COMPRISING HUMANIZED ANTIBODIES TARGETING UROKINASE TYPE PLASMINOGEN ACTIVATOR RECEPTOR ASSOCIATED PROTEIN (UPARAP)

(71) Applicants: RIGSHOSPITALET, København Ø (DK); UNIVERSITY OF COPENHAGEN, Copenhagen K (DK)

(72) Inventors: Christoffer NIELSEN, Hillerød (DK); Niels Behrendt, Vaerløse (DK); Lars Henning ENGELHOLM, Bagsværd (DK)

(21) Appl. No.: 18/565,621

(22) PCT Filed: Jun. 29, 2022
(86) PCT No.: PCT/EP2022/067832

§ 371 Date: Nov. 30, 2023

(30) Foreign Application Priority Data
Jun. 29, 2021 (EP) 21182271.3

(51) Int. Cl. A61K 47/68; A61K 51/10; C07K 16/28

(52) U.S. Cl. CPC A61K 47/68037; A61K 47/6849; A61K 47/6889; A61K 51/1027; A61K 51/1093; C07K 16/2851; A61K 2121/00; C07K 2317/56

(57) ABSTRACT

The present invention relates to humanized antibodies and molecular conjugates targeting Urokinase type plasminogen activator receptor associated protein (uPARAP), in particular antibody-drug conjugates (ADCs) comprising humanized antibodies directed against uPARAP and their use in delivery of active agents to cells and tissues expressing uPARAP. The invention further relates to the use of said ADCs in the treatment of diseases involving uPARAP expressing cells, such as certain cancers.

### CROSS-REFERENCE TO RELATED APPLICATIONS

This application is the National Stage Application of International Patent Application No. PCT/EP2022/067832, filed on Jun. 29, 2022, which claims the benefit of European Patent Application Number 21182271.3, filed on Jun. 29, 2021, the disclosures of each of which are herein incorporated by reference in their entireties.

### REFERENCE TO SEQUENCE LISTING SUBMITTED ELECTRONICALLY

The contents of the electronic sequence listing (105728_000106_SEQUENCE_LISTING; Size: 29,183 bytes; and Date of Creation: Nov. 29, 2023) is herein incorporated by reference in its entirety.

### FIELD OF INVENTION

The present invention relates to antibodies and molecular conjugates targeting the receptor uPARAP, in particular antibody-drug conjugates (ADCs) comprising humanized antibodies directed against uPARAP and their use in delivery of active agents to cells and tissues expressing uPARAP. The invention further relates to the use of said ADCs in the treatment of diseases involving uPARAP expressing cells, such as certain cancers.

### BACKGROUND

Urokinase-type Plasminogen Activator Receptor Associated Protein (uPARAP), also known as CD280, Endo180 and mannose receptor C type 2, is a member of the macrophage mannose receptor family of endocytic transmembrane glycoproteins. uPARAP is a membrane protein involved in matrix turnover during tissue remodelling, particularly the uptake and intracellular degradation of collagen. The uPARAP receptor consists of an N-terminal cysteine-rich domain (CysR), a fibronectin type II (FN-II) domain, and eight C-type lectin-like domains (CTLDs 1-8)

The receptor uPARAP is upregulated in the tumour cells of specific cancers, including sarcomas and late-stage glioblastoma. Additionally, the receptor is most often upregulated in stromal cells surrounding solid tumours and some literature suggests a high expression of uPARAP in bone metastasis from prostate cancer (Caley et al., 2012, J. Pathol 5: 775-783). In healthy adult individuals, the receptor displays a restricted expression pattern (Melander et al., 2015, Int J Oncol 47: 1177-1188).

Antibody-drug conjugates (ADCs) are a class of highly potent biopharmaceutical drugs designed as a targeted therapy, in particular for the treatment of cancer. ADCs are complex molecules composed of an antibody (a whole mAb or an antibody fragment) linked, via a stable, chemical, linker that may possess labile bonds, to an active agent, such as a biologically active drug or cytotoxic compound. By combining the unique targeting capabilities of antibodies with the cell-killing ability of cytotoxic drugs, antibody-drug conjugates allow sensitive discrimination between healthy and diseased tissue, based on expression of the antibody antigen. This means that, in contrast to traditional chemotherapeutic agents, antibody-drug conjugates actively target and attack cancer cells, so that healthy cells with little or no antigen expression are less severely affected. To date, more than 10 ADCs have received market approval and several ADCs are currently in clinical trials.

WO 2010/111198 discloses conjugates comprising an anti-uPARAP antibody and suggests use of such conjugates in the delivery of therapeutic agents to cells that express uPARAP.

WO 2017/133745 discloses ADCs directed against uPARAP.

Treatment methods currently exist for most cancer types. However, in many cases with unsatisfactory efficiency or with adverse effects due to high dosing of the therapeutic agent. Thus, there is a need for more efficient treatments with increased potency.

### SUMMARY

Provided herein is a humanized version of the murine 9b7 antibody, and its implementation in antibody-drug conjugates (ADCs) targeting the uPARAP receptor. The murine 9b7 antibody was originally described in WO 2017/133745. The antibodies and ADCs as described herein are capable of specifically targeting cells and tissues expressing uPARAP, and demonstrate enhanced efficacy compared to ADCs comprising the murine 9b7 antibody as well as enhanced efficacy compared to other humanized versions of the murine 9b7 antibody.

In particular, the present disclosure relates to an antibody which binds to uPARAP comprising:
a. an immunoglobulin light chain variable region comprising or consisting of the amino acid sequence of SEQ ID NO: 3; and/or
b. an immunoglobulin heavy chain variable region comprising or consisting of the amino acid sequence of SEQ ID NO: 6.

Further, the present disclosure relates to an antibody-drug conjugate (ADC) comprising:
a. the antibody as defined herein above,
b. an active agent, and
c. optionally a linker which links a) to b).

Furthermore, the present disclosure relates to a method for treatment of a disease characterised by cells expressing uPARAP, said method comprising administering to a subject the antibody as defined herein above, the ADCs as defined herein above, or a pharmaceutical composition comprising the antibody or the ADCs as defined herein above.

Further aspects of the present disclosure are a polypeptide comprising or consisting of the amino acid sequence of SEQ ID NO: 2 and/or SEQ ID NO: 5; an isolated polynucleotide encoding the amino acid sequence as defined herein; a vector comprising the polynucleotide as defined above; and a host cell comprising the polynucleotide as defined above and/or the vector as defined above.

An even further aspect of the present disclosure is a kit comprising the antibody as defined above, the ADCs as defined above, or a pharmaceutical composition comprising the antibody or the ADCs as defined herein above, optionally further comprising means for administering the antibody or antibody-drug conjugate to a subject and/or instructions for use

### DESCRIPTION OF DRAWINGS

FIG. 1: In vitro cell viability assays of U937 cancer cell lines exposed to MMAE-based ADCs comprising either the LC0HC0 antibody (comprising the variable domains of the original murine 9b7 antibody fused to human IgG constant regions), or the humanized LC4HC3 antibody. But for the antibody, the two ADCs are identical and were produced by identical methods. Cells were incubated for 96 hours, before being analyzed by colorimetric viability assay. The assay for the U937 cell line shows that ADCs based on LC4HC3 have a significantly greater reduction in overall cell viability compared to the LC0HC0 ADCs.

FIG. 2: Internalization of humanized antibodies LC4HC3 and LC3HC3 in SAOS-2 osteosarcoma cells. Detailed protocols are presented in Example 2. The data shows that LC4HC3 is internalized not only faster than LC3HC3 but also to a greater extent in SAOS-2 osteosarcoma cells.

FIG. 3: In vivo efficacy of Vedotin-type ADCs based on LC4HC3 (LC4HC3-vc-MMAE, FIG. 3a) and LC3HC3 (LC3HC3-vc-MMAE, FIG. 3b). CB17 mice were inoculated with U937 cells to induce tumor growth. Tumor size was closely monitored and treatment initiated once a size of approximately 80-150 mm³ was reached. But for the antibody, the two ADCs are identical and were produced by identical methods. Each line in FIGS. 3a and 3b represents tumor size in a mouse administered a 4 mg/kg dose of the referenced ADC for 7 days, twice daily. The data shows that ADCs based on humanized 9b7 antibody LC4HC3 are superior antitumor agents compared to ADCs based on a different humanized 9b7 antibody, LC3HC3.

### DETAILED DESCRIPTION

The antibodies of the present disclosure are internalised upon binding to uPARAP receptors at the cell surface, thus allowing for intracellular actions of the active agent of the antibody-drug conjugate complex.

Provided herein are humanised versions of the murine 9b7 antibody, which bind to the uPARAP receptor.

### Anti-uPARAP Humanised Antibodies

Methods of generating antibodies are well known in the art. For example, antibodies may be generated via any one of several methods which employ induction of in vivo production of antibody molecules, screening of immunoglobulin libraries, or generation of monoclonal antibody molecules by cell lines in culture. These include, but are not limited to, the hybridoma technique, the human B-cell hybridoma technique, and the Epstein-Barr virus (EBV)-hybridoma technique.

Humanised antibodies are generally preferred in medicines intended for humans and methods for humanising antibodies are well known in the art. Although humanisation techniques are known, it can be a challenge to achieve humanised antibodies that retain the binding properties of the initial antibody and even more challenging to achieve humanised antibodies with improved characteristics, such as improved ligand affinity and efficacy compared to the initial antibody.

The inventors herein provide an improved anti-uPARAP antibody, which is a humanised version of the 9b7 murine antibody and which displays improved ligand affinity and efficacy compared to the 9b7 murine antibody as well as improved internalization and in vivo efficacy compared to other humanized versions of the 9b7 antibody.

The anti-uPARAP antibody of the present disclosure may be of any immunoglobulin class including IgG, IgM, IgD, IgE, IgA, and any subclass thereof. IgG subclasses are also well known to those in the art and include but are not limited to human IgGI, IgG2, IgG3 and IgG4. In one embodiment the antibody is an IgG monoclonal antibody. In one embodiment the antibody is IgG1 K.

The anti-uPARAP antibody of the present disclosure is a humanised 9b7 antibody, which binds to the uPARAP receptor, more specifically, the humanized 9b7 antibody disclosed herein binds at least to the fibronectin type II (FN-II) domain of the uPARAP receptor.

The humanized 9b7 antibody, also referred to herein as 980.2 LC4HC3, comprises a light chain variable region of amino acids comprising SEQ ID NO: 3, which is the variable region of LC4, and a heavy chain variable region of amino acids comprising SEQ ID NO:6, which is the variable region of HC3.

The humanized 9b7 antibody, also referred to herein as 980.2 LC4HC3, may comprise a light chain of amino acids comprising or consisting of SEQ ID NO: 1, which is LC4, and a heavy chain of amino acids comprising or consisting of SEQ ID NO:4, which is HC3.

In one embodiment of the present disclosure, the anti-uPARAP antibody as defined herein comprises:
a. an immunoglobulin light chain variable region comprising or consisting of the amino acid sequence of SEQ ID NO: 3; and/or
b. an immunoglobulin heavy chain variable region comprising or consisting of the amino acid sequence of SEQ ID NO: 6.

In one embodiment of the present disclosure, the antibody which binds to uPARAP as defined herein comprises:
a. an immunoglobulin light chain comprising the amino acid sequence of SEQ ID NO: 1 (LC4); and/or
b. an immunoglobulin heavy chain comprising the amino acid sequence of SEQ ID NO: 4 (HC3).

In one embodiment of the present disclosure, the antibody which binds to uPARAP as defined herein comprises:
a. an immunoglobulin light chain consisting of the amino acid sequence of SEQ ID NO: 1 (LC4); and
b. an immunoglobulin heavy chain consisting of the amino acid sequence of SEQ ID NO: 4 (HC3).

### Polypeptides, Polynucleotides, Vectors and Host Cells

One embodiment of the present disclosure is a polypeptide comprising or consisting of the amino acid sequence of SEQ ID NO: 2 and/or SEQ ID NO: 5. SEQ ID NO: 2 and SEQ ID NO: 5 correspond to SEQ ID NO: 1 and SEQ ID NO: 4, respectively, but further have a N-terminal signal peptide for expression purposes.

One embodiment of the present disclosure is an isolated polynucleotide encoding any of the polypeptides disclosed herein, i.e. an isolated polynucleotide which encodes the amino acid sequence of any one of SEQ ID NOs: 1, 2, 3, 4, 5 and/or 6.

In one embodiment, the polynucleotide comprises SEQ ID NO: 11 and/or SEQ ID NO: 12, encoding SEQ ID NO: 2 and SEQ ID NO: 5, respectively.

In one embodiment the polypeptide comprises or consists of the amino acid sequence of SEQ ID NO: 2, optionally wherein the polypeptide further comprises the amino acid sequence of SEQ ID NO: 5.

In one embodiment this disclosure provides an isolated polynucleotide which encodes the amino acid sequence of any one of SEQ ID NOs: 1, 2, or 3, optionally wherein the polynucleotide further encodes the amino acid sequence of any one of 4, 5 or 6.

In one embodiment this disclosure provides an isolated polynucleotide comprising SEQ ID NO: 11, optionally wherein the polynucleotide further comprises SEQ ID NO: 12.

In one embodiment the polypeptide is an isolated polypeptide.

One embodiment of the present disclosure is a vector, such as an expression vector, comprising the polynucleotide as defined herein.

In one embodiment of the present disclosure, the vector is a mammalian expression vector.

In one embodiment of the present disclosure, the vector is a plasmid vector, such as a plasmid vector selected from pD2610-v13 (ATUM), pSV and the pCMV series of plasmid vectors.

In one embodiment of the present disclosure, the vector is a viral vector, such as a viral vector selected from the group consisting of adenoviral vectors, lentiviral vectors, adeno-associated viral vectors, herpesviral vectors, vaccinia viral vectors, poxviral vectors, baculoviral vectors and oncolytic viral vectors.

A further embodiment of the present disclosure is a host cell comprising the polynucleotide and/or the vector as defined herein.

In one embodiment of the present disclosure, the host cell comprising the polynucleotide and/or the vector as described herein is selected from the group consisting of CHO (Chinese hamster ovary) cells, COS (CV-1 (simian) in Origin, and carrying the SV40 genetic material) cells, HEK (Human embryonic kidney) cells and HeLa (Henrietta Lacks) cells.

In one embodiment, the host cell is CHO.

In one embodiment the host cell is a recombinant host cell.

### Antibody-Drug Coniugates (ADCs) Comprising Anti-uPARAP Humanised Antibodies

The data of the inventors surprisingly shows that ADCs based on LC4HC3 (humanized 9b7 antibody) result in a significantly greater reduction in overall cell viability compared to the ADCs based LC0HC0 (having variable domains of the 9b7 murine antibody fused to human IgG constant regions). ADCs based on LC4HC3 also exhibit improved internalization and in vivo efficacy compared to ADCs based on LC3HC3 (another humanized 9b7 antibody).

One particularly preferred embodiment of the present disclosure is an antibody-drug conjugate (ADC) comprising:
a. the antibody as defined herein,
b. an active agent, and
c. optionally a linker which links a) to b).

In one embodiment of the present disclosure, the antibody-drug conjugate (ADC) as defined herein comprises:
a. the antibody as defined herein, comprising:
   i) an immunoglobulin light chain variable region comprising or consisting of the amino acid sequence of SEQ ID NO: 3; and/or
   ii) an immunoglobulin heavy chain variable region comprising or consisting of the amino acid sequence of SEQ ID NO: 6
b. an active agent, and
c. optionally a linker which links a) to b).

In one embodiment of the present disclosure, the antibody-drug conjugate (ADC) as defined herein comprises:
a. the antibody as defined herein, comprising:
   i) an immunoglobulin light chain comprising or consisting of the amino acid sequence of SEQ ID NO: 1; and/or
   ii) an immunoglobulin heavy chain comprising or consisting of the amino acid sequence of SEQ ID NO: 4
b. an active agent, and
c. optionally a linker which links a) to b).

In one embodiment of the present disclosure, the antibody-drug conjugate (ADC) as defined herein comprises:
a. the antibody as defined herein, comprising:
   i) an immunoglobulin light chain consisting of the amino acid sequence of SEQ ID NO: 1; and
   ii) an immunoglobulin heavy chain consisting of the amino acid sequence of SEQ ID NO: 4
b. an active agent, and
c. optionally a linker which links a) to b).

### Active Agent

The ADCs of the present disclosure comprise an active agent, e.g. a drug, which can be delivered intracellularly to cells expressing uPARAP. The active agent may e.g. be a therapeutic agent, a radioisotope or a detectable label. In a preferred embodiment the active agent is a therapeutic agent.

In one embodiment, the active agent may be or comprise a radioisotope. The radioisotope may serve as a radiation emitter either for treatment of affected tissues or for diagnostic purposes. In one embodiment, the radioisotope may consist of or comprise ⁶⁰Co, ⁸⁹Sr, ⁹⁰Y, ^{99m}Tc, ¹³¹I, ¹³⁷Cs, ¹⁵³Sm, or ²²³Rd. In one embodiment of the present disclosure, the radioisotope may be in combination with a chelator such as DOTA or EDTA or others which are well known in the art.

In one embodiment the active agent is a therapeutic agent. Classes of therapeutic agents include DNA crosslinking agents, DNA alkylating agents, DNA strand scission agents, anthracyclines, antimetabolites, anti-microtubule/anti-mitotic agents, histone deacetylase inhibitors, kinase inhibitors, metabolism inhibitors, peptide antibiotics, immune checkpoint inhibitors, platinum-based antineoplastics, topoisomerase inhibitors, DNA or RNA polymerase inhibitors, nucleotide based agents, and cytotoxic antibiotics.

In a preferred embodiment the active agent is a cytotoxic agent allowing for efficient killing of the cells expressing uPARAP.

In one embodiment the active agent is a chemotherapeutic agent.

In one embodiment, the active agent is a DNA-crosslinking agent, such as a DNA crosslinking agent selected from cisplatin or a derivative of cisplatin such as carboplatin or oxaliplatin, mitomycin C (MMC), pyrrolobenzodiazepine, and dimeric pyrrolobenzodiazepine derivatives such as SGD-1882 or a derivative of any of these.

In one embodiment of the present disclosure, the active agent is a DNA alkylating agent, such as a DNA alkylating agent selected from nitrogen mustards such as tris(2-chloroethyl)amine, pyridinobenzodiazepines or a pyridinobenzodiazepine derivative, indolinobenzodiazepine dimers, and Duocarmycin SA or a derivative of any of these.

In one embodiment, the active agent is a DNA strand scission agent, such as a DNA strand scission agent selected from calicheamicin and hamiltrone or a derivative of any of these.

In one embodiment the active agent is an anthracycline, such as an anthracycline selected from Daunorubicin, doxorubicin, epirubicin, idarubicin, and PNU-159682 or a derivative of any of these.

In one embodiment the active agent is an antimetabolite, such as an antimetabolite selected from folic acid antagonists such as methotrexate, purine antimetabolites such as 6-mercaptopurine or 6-thioguanine or fludarabine phosphate or pentostatin or cladribine, and pyrimidine antimetabolites such as 5-fluorouracil or 5-fluorodeoxyuridine or cytarabine or gemcitabine, or a derivative of any of these.

In one embodiment the active agent is an anti-mitotic agent, such as selected from the group consisting of derivatives of auristatin or dolastatin such as monomethyl auristatin E (MMAE), monomethyl auristatin F (MMAF) and more, a taxane such as Paclitaxel or Docetaxel and more, a vinca alkaloid such as Vinblastine, Vincristine, Vindesine or Vinorelbine and more, a mayatansinoid, Colchicine, and Podophyllotoxin or a derivative of any of these.

In one embodiment, the active agent is monomethyl auristatin E (MMAE) or a derivative thereof.

Because of its high toxicity, MMAE, which inhibits cell division by blocking the polymerization of tubulin, cannot be used as a single-agent chemotherapeutic drug. However, the combination of MMAE linked to an anti-CD30 monoclonal antibody (Brentuximab Vedotin, trade name Adcetris™) has been proven to be stable in extracellular fluid, cleavable by cathepsin and safe for therapy.

In one embodiment, the active agent is a histone deacetylase inhibitor, such as a histone deacetylase inhibitor selected from trichostatin A, vorinostat, belinostat, panabiostat, givinostat, resminostat, abexinostat, quisinostat, rocilinostat, practinostat, CHR-3996, valproic acid, butyric acid, phenylbutyric acid, entinostat, tacedinaline, 4SC202, mocetinostat, romidepsin, nicotinamide, sirtinol, cambinol, and EX-527 or a derivative of any of these.

In one embodiment, the active agent is a kinase inhibitor, such as a kinase inhibitor selected from genistein, lavendustin C, PP1-AG1872, PP2-AG1879, SU6656, CGP77675, PD166285, imatinib, erlotinib, gefitinib, lavendustin A, cetuximab, UCS15A, herbimycin A, and radicicol or a derivative of any of these.

In one embodiment, the active agent is a metabolism inhibitor, such as an NAMPT inhibitor. Examples of NAMPT inhibitors include AP0866, GMX-1777, GMX-1778 ATG-019, and OT-82 or a derivative of any of these.

In one embodiment, the active agent is an immune checkpoint inhibitor, such as a PD-1 inhibitor or a PD-L1 inhibitor. Examples of PD-1 inhibitors include Pembrolizumab, Nivolumab, Cemiplimab, JTX-4014, Spartalizumab, Camrelizumab, Sintilimab, Tislelizumab, Toripalimab, Dostarlimab, AMP-224 and AMP-514. Examples of PD-L1 inhibitors include Atezolizumab, Avelumab, Durvalumab, KN035, CK-301, AUNP12, CA-170 and BMS-986189 or a derivative of any of these.

In one embodiment, the active agent is a platinum-based antineoplastic, such as a platinum-based antineoplastic selected from lipoplatin, cisplatin, carboplatin, oxaliplatin, nedaplatin, picoplatin, phenanthriplatin, satraplatin, and triplatin tetranitrate or a derivative of any of these.

In one embodiment, the active agent is a topoisomerase inhibitor, such as a topoisomerase inhibitor selected from camptothecin or derivatives thereof such as topotecan, belotecan, lurtotecan, irinotecan, SN-38, exatecan, and Dxd or a derivative ofany ofthese.

In one embodiment, the active agent is a DNA- or RNA-polymerase inhibitor, such as a polymerase inhibitor selected from amanitin or alpha-amanitin or derivatives thereof, actinomycin D, and aphidicolin or a derivative of any of these.

In one embodiment, the active agent is a nucleotide-based agent, such as an RNA- or DNA-oligonucleotide, such as an siRNA or a miRNA.

There may be one or more units of drug per antibody molecule. The ratio between the number of drug molecules per antibody is denoted the drug-to-antibody ratio (DAR). In one embodiment, the DAR is between 1 and 10, such as between 2 and 8, for example between 2 and 6, such as 2 or 4.

### Linker

A stable link between the antibody and the active agent is an important aspect of ADC technology. Linkers may e.g. be based on chemical motifs including disulfides, hydrazones or peptides (cleavable), or thioethers (noncleavable), and control the distribution and delivery of the cytotoxic agent to the target cell. Cleavable and noncleavable types of linkers have been proven to be safe in preclinical and clinical trials. For example, Brentuximab Vedotin includes an enzyme-sensitive cleavable linker that delivers the potent and highly toxic antimicrotubule agent monomethyl auristatin E (MMAE), a synthetic antineoplastic agent, to cells.

Trastuzumab Emtansine, another approved ADC, is a combination of the microtubule-formation inhibitor mertansine (DM-1), a derivative of the Maytansine, and antibody Trastuzumab (Herceptin™, Genentech/Roche), attached by a stable, non-cleavable linker.

The type of linker, cleavable or non-cleavable, lends specific properties to the delivered drug. For example, cleavable linkers can e.g. be cleaved by enzymes in the target cell, leading to efficient intracellular release of the active agent, for example a cytotoxic agent. In contrast, an ADC containing a non-cleavable linker has no mechanism for drug release, and must rely on mechanisms such as degradation of the targeting antibody, for drug release. Furthermore, as is appreciated by those skilled in the art, the linker composition may influence critical factors such as solubility and pharmacokinetic properties of the ADC as a whole.

For both types of linker, drug release is crucial for obtaining a cellular effect. Drugs which are able to freely diffuse across cell membranes may escape from the targeted cell and, in a process called “bystander killing,” also attack neighbouring cells, such as cancer cells in the vicinity of the uPARAP expressing target cell.

In a preferred embodiment of the present disclosure, the ADC targeting uPARAP as disclosed herein comprises a linker that links the antibody to the active agent.

In one embodiment of the present disclosure, the linker may be cleavable or non-cleavable.

Cleavable groups include a disulfide bond, an amide bond, a substituted amide bond in the form of a peptide bond, a thioamide, bond, an ester bond, a thioester bond, a vicinal diol bond, or a hemiacetal. These, or other cleavable bonds, may include enzymatically-cleavable bonds, such as peptide bonds (cleaved by peptidases), phosphate bonds (cleaved by phosphatases), nucleic acid bonds (cleaved by endonucleases), and sugar bonds (cleaved by glycosidases).

In a further embodiment of the present disclosure, the linker is a cleavable linker allowing for intracellular release of the active agent inside the target cells.

In a further embodiment the linker is a peptide linker. The choice of peptide sequence is critical to the success of the conjugate. In some embodiments the linker is stable to serum proteases, yet is cleaved by lysosomal enzymes in the target cell.

In a further embodiment the linker is an enzyme-cleavable peptide-containing linker, such as a cathepsin cleavable peptide-containing linker. Cathepsin can be one of several cathepsin types, being one of a group of lysosomal proteases.

In a further embodiment of the present disclosure, the linker comprises or consists of a dipeptide, such as valine-citrulline (VC) or valine-alanine (VA).

In one embodiment the linker comprises or consists of a dipeptide, such as valine-citrulline (VC) or valine-alanine (VA), which may be further connected through an amide linkage to other structural elements. Valine-citrulline-based linkers, in which the citrulline carboxyl function is modified to a substituted amide, can be cleaved by lysosomal cathepsins, whereas valine-alanine-based linkers, in which the alanine carboxyl function is modified to a substituted amide, can be cleaved by other lysosomal proteases, including other cathepsins.

In a further embodiment of the present disclosure, the antibody-drug conjugate as defined herein further comprises a spacer, such as a spacer comprising p-aminobenzoic acid (PAB), p-aminobenzylcarbamate (PABC), p-aminobenzoyloxycabonyl, or polyethylenglycol (PEG).

In one embodiment of the present disclosure, the antibody-drug conjugate as defined herein comprises p-aminobenzylcarbamate (PABC).

In a further embodiment of the present disclosure, the antibody-drug conjugate as defined herein further comprises an attachment group, such as an attachment group comprising or consisting of maleimide and caproic acid (MC), N-hydroxysuccinimide, reactive attachment groups directed to modified or unmodified protein-bound carbohydrate, peptide sequences that are required for enzymatic reactions, azides or alkynes or being derived from these by reaction with the antibody or a chemically or enzymatically generated derivative thereof.

In one embodiment of the present disclosure, the ADC of the present disclosure further comprises an attachment entity. The attachment entity may for example connect the antibody and the cleavable linker, where the attachment entity is the reaction product between an antibody amino acid side chain and a reactive attachment group in the linker precursor. In one embodiment, this reactive attachment group comprises or consists of maleimide and caproic acid (MC), where maleimide reacts preferably with cysteine thiols during coupling. In other embodiments, the attachment group comprises or consists of N-hydroxysuccinimide, reactive attachment groups directed to modified or unmodified protein-bound carbohydrate, peptide sequences that are required for enzymatic reactions, azides or alkynes or being derived from these by reaction with the antibody or a chemically or enzymatically generated derivative thereof.

In one embodiment of the present disclosure, the ADC comprises an antibody targeting uPARAP as defined herein, and the linker-drug complex Vedotin. Vedotin is a linker-drug complex comprising the cytotoxic agent MMAE, a spacer (p-aminobenzoic acid), a cathepsin-cleavable linker (Valine-citrulline dipeptide) and an attachment group consisting of caproic acid and maleimide. Vedotin is MC-VC-PAB-MMAE.

In one embodiment, the ADC of the present disclosure targeting uPARAP comprises the antibody as defined herein, and a linker-spacer-toxin unit being VC-PAB-MMAF.

In one embodiment, the ADC of the present disclosure targeting uPARAP comprises the antibody as defined herein, and a linker-spacer-toxin unit being VC-PABC-MMAF.

In one embodiment, the ADC of the present disclosure targeting uPARAP comprises or consists of:
a. the antibody as defined herein, comprising:
   i) an immunoglobulin light chain consisting of the amino acid sequence of SEQ ID NO: 1; and
   ii) an immunoglobulin heavy chain consisting of the amino acid sequence of SEQ ID NO: 4.
b. a VC linker,
c. an MC attachment group,
d. a PAB or a PABC spacer, and
e. MMAE as active agent.

In one embodiment, the ADC of the present disclosure targeting uPARAP comprises or consists of:
a. the antibody as defined herein, comprising:
   i) an immunoglobulin light chain variable region comprising or consisting of the amino acid sequence of SEQ ID NO: 3; and/or
   ii) an immunoglobulin heavy chain variable region comprising or consisting of the amino acid sequence of SEQ ID NO: 6,
b. a VC linker,
c. an MC attachment group,
d. a PAB or a PABC spacer, and
e. MMAE as active agent.

### Therapeutic Use

The ADCs directed against uPARAP as described herein are useful for the delivery of active agents, such as therapeutic or cytotoxic agents to cells expressing uPARAP and similar proteins and thus for the treatment of a range of diseases and disorders characterized by either expression or overexpression of said proteins.

Thus, one embodiment of the present disclosure is the antibody or the antibody-drug conjugate as defined herein for use as a medicament.

One embodiment of the present disclosure is a pharmaceutical composition comprising an effective amount of the antibody or the antibody-drug conjugate as defined herein, and a pharmaceutically acceptable buffer, diluent, carrier, adjuvant or excipient.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce, and most preferably prevent, a clinically significant deficit in the activity, function and response of the host. Alternatively, a therapeutically effective amount is sufficient to cause an improvement in a clinically significant condition in a host.

As is appreciated by those skilled in the art, the amount of a compound may vary depending on its specific activity. Suitable dosage amounts may contain a predetermined quantity of active composition calculated to produce the desired therapeutic effect in association with the required diluent.

The ADCs of the present disclosure may be formulated into any type of pharmaceutical composition known in the art to be suitable for the delivery thereof.

The pharmaceutical compositions may be prepared in a manner known in the art that is sufficiently storage stable and suitable for administration to humans and/or animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the ADC. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated herein by reference).

The term “buffer” is intended to mean an aqueous solution containing an acid-base mixture with the purpose of stabilising pH. Pharmaceutically acceptable buffers are well known in the art.

The term “diluent” is intended to mean an aqueous or non-aqueous solution with the purpose of diluting the agent in the pharmaceutical preparation.

The term “adjuvant” is intended to mean any compound added to the formulation to increase the biological effect of the agent of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

Another embodiment of the present disclosure is a method for treatment of a disease characterised by cells in a subject expressing uPARAP, said method comprising administering to the subject the antibody or the antibody-drug conjugate as defined herein.

The expression and role of uPARAP in cancer has been investigated by several research groups; cf. review by Melander et al (Melander et al., 2015, Int J Oncol 47: 1177-1188) and article by Engelholm et al (Engelholm et al., 2016, J. Pathol. 238, 120-133).

In one embodiment of the present disclosure, the method is the method as defined herein, wherein the disease characterised by cells expressing uPARAP is selected from cancer, a bone degradation disease such as osteoporosis, fibrosis, and macrophage associated diseases or disorders such as atherosclerosis, arthritis, or chronic inflammation.

In one embodiment of the present disclosure, the method is the method as defined herein, wherein the arthritis is selected from osteoarthritis, inflammatory arthritis, rheumatoid arthritis, psoriatic arthritis, lupus, Lyme disease-induced arthritis such as Lyme arthritis, gout or pseudogout, and ankylosing spondylitis.

In one embodiment of the present disclosure, the method is the method as defined herein, wherein the disease is cancer.

Examples of cancers characterized by overexpression of uPARAP include, but are not limited to, sarcoma, including osteosarcoma (Engelholm et al., 2016, J Pathol 238(1): 120-33) as well as other sarcomas, glioblastoma (Huijbers et al., 2010, PLoS One 5(3): e9808), prostate cancer and bone metastases from prostate cancer (Kogianni et al., 2009, Eur J Cancer 45(4): 685-93), breast cancer and in particular “basal like” breast cancer (Wienke et al., 2007, Cancer Res 1; 67(21): 10230-40), head- and neck cancer (Sulek et al., 2007, J Histochem Cytochem 55(4): 347-53), and mesothelioma (Çakilkayaet al., 2021, Int J Mol Sci 22(21): 11452).

In one embodiment of the present disclosure, the method is the method as defined herein, wherein the cancer is selected from sarcoma, glioblastoma, mesothelioma, colon cancer, prostate cancer, bone metastases from prostate cancer, breast cancer, head- and neck cancer, and leukaemia.

In one embodiment of the present disclosure, the method is the method as defined herein, wherein the cancer is leukaemia, such as acute lymphoblastic leukaemia (ALL), acute myeloid leukaemia (AML), chronic lymphocytic leukaemia (CLL), and chronic myeloid leukaemia (CML), or subtypes of these.

In one embodiment of the present disclosure, the method is the method as defined herein, wherein the cancer is sarcoma, such as osteosarcoma, or soft tissue sarcoma (STS), or subtypes of these.

In one embodiment of the present disclosure, the method is the method as defined herein, the soft tissue sarcoma (STS) is selected from epithelioid sarcoma, clear cell sarcoma, alveolar soft part sarcoma, extraskeletal myxoid chondrosarcoma, epithelioid hemangioendothelioma, inflammatory myofibroblastic tumor, undifferentiated embryonal sarcoma, alveolar soft part sarcoma (ASPS), angiosarcoma, chondrosarcoma, dermatofibrosarcoma protuberens (DFSP), desmoid sarcoma, Ewing's sarcoma, fibrosarcoma, myxofibrosarcome, gastrointerstinal stromal tumor (GIST), non-uterine leiomyosarcoma, uterine leiomyosarcoma, liposarcoma, malignant fibro histiocytoma (MFH), malignant peripheral nerve sheath tumor (MPNST), rhabdomyosarcoma, synovial sarcoma, and/or leiomyosarcoma (LMS).

In one embodiment of the present disclosure, the method is the method as defined herein, wherein the cancer is metastatic cancer

In one embodiment of the present disclosure, the method is the method as defined herein, wherein the cancer is a solid tumour.

In one embodiment of the present disclosure, the method is the method as defined herein, wherein the cancer is glioblastoma.

In one embodiment of the present disclosure, the cancer is not a solid tumour. For instance, the ADC of the present disclosure may e.g. be used for the treatment of uPARAP-expressing leukemia, for example, from the macrophage-monocyte lineage.

In other embodiments of the present disclosure, the disease or disorder characterised by cells expressing uPARAP is not cancer.

uPARAP is involved in bone growth and homeostasis (Madsen et al., 2013, PLoS One 5; 8(8): e71261). Thus, in one embodiment the ADC of the present disclosure may be used for the treatment of a disease characterized by bone degradation, wherein the bone degradation is mediated by non-malignant cells, such as osteoporosis.

Due to its role in collagen accumulation, a role for uPARAP has also been shown in fibrosis (Madsen et al., 2012, J Pathol 227(1):94-105). Thus, in one embodiment the ADC of the present disclosure may be used for the treatment of fibrosis, for example of kidney, lung and liver.

In one embodiment of the present disclosure, the ADC of the present disclosure may be used for the treatment of diseases and disorders associated with macrophages, including atherosclerosis, arthritis, and chronic inflammation.

The ADCs of the present disclosure or pharmaceutical compositions comprising the ADCs may be administered via any suitable route known to those skilled in the art. Thus, possible routes of administration include parenteral (intravenous, subcutaneous, and intramuscular), topical, ocular, nasal, pulmonar, buccal, oral, vaginal and rectal. Also, administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered if necessary.

In one embodiment of the present disclosure, the method is the method as defined herein, wherein the antibody-drug conjugate is administered parenterally, for example, intravenously, intracerebroventricularly, intraarticularly, intra-arterially, intraperitoneally, intrathecally, intraventricularly, intrasternally, intracranially, intramuscularly or subcutaneously, or by infusion techniques.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

In one embodiment of the present disclosure, the method is the method as defined herein, wherein the antibody-drug conjugate or the antibody is administered intravenously.

In one embodiment of the present disclosure, the method is the method as defined herein, wherein the antibody-drug conjugate or the antibody is administered subcutaneously.

In one embodiment of the present disclosure, the method is the method as defined herein, wherein the antibody-drug conjugate or the antibody is administered in combination with one or more further agents, such as one or more further therapeutic agents.

In one embodiment of the present disclosure, the ADC or the antibody of the present disclosure is administered in conjunction with additional reagents and/or therapeutics that may increase the functional efficiency of the ADC, such as established or novel drugs that increase lysosomal membrane permeability, thereby facilitating molecular entry from the lysosome interior to the cytoplasm, or drugs that increase the permeability of the blood-brain barrier.

In one embodiment of the present disclosure, the ADCs or the antibodies described herein may be administered in combination with a range of anti-cancer agents, such as antimetabolites, alkylating agents, anthracyclines and other cytotoxic antibiotics, vinca alkyloids, anti-microtubule/anti-mitotic agents, histone deacetylase inhibitors, kinase inhibitors, peptide antibiotics, immune checkpoint inhibitors, platinum-based antineoplastics, etoposide, taxanes, topoisomerase inhibitors, antiproliferative immunosuppressants, corticosteroids, sex hormones and hormone antagonists, cytotoxic antibiotics and other therapeutic agents.

Thus, in one embodiment of the present disclosure, the method is the method as defined herein, wherein the cell expressing uPARAP displays uPARAP overexpression.

In one embodiment of the present disclosure, the method is the method as defined herein, wherein the cell expressing uPARAP is a tumour cell.

In one embodiment of the present disclosure, the method is the method as defined herein, wherein the cell expressing uPARAP is a tumour associated cell.

Tumour associated cells include, but are not limited to, activated fibroblasts, myofibroblasts, neovasculature and infiltrating cells of the macrophage-monocyte lineage or other leukocytic cell types, as well as cells of the stromal tissue surrounding the tumour.

In one embodiment of the present disclosure, the method is the method as defined herein, wherein the antibody or the antibody-drug conjugate induces cell death and/or inhibits the growth and/or proliferation of the uPARAP expressing cell.

In one embodiment of the present disclosure, the method is the method as defined herein, wherein the antibody or the antibody-drug conjugate induces liberation of free cytotoxin from the uPARAP expressing cells, leading to cell death and/or inhibition of the growth and/or proliferation of neighbouring cancer cells.

In one embodiment of the present disclosure, the method is the method as defined herein, wherein the treatment is ameliorative or curative.

A further embodiment of the present disclosure is a method for inhibiting tumour progression in a subject, comprising administering to the subject the antibody or the antibody-drug conjugate or the pharmaceutical composition as defined herein to said subject.

A further embodiment of the present disclosure is a method for inhibiting, lowering or eliminating metastatic capacity of a tumour in a subject, comprising administering to the subject the antibody or the antibody-drug conjugate or the pharmaceutical composition as defined herein to said subject.

An even further embodiment of the present disclosure is a kit comprising the antibody or the antibody-drug conjugate or the pharmaceutical compositions as defined herein, optionally further comprising means for administering said antibody-drug conjugate or pharmaceutical composition to a subject and/or instructions for use.

In one embodiment, the present disclosure relates to an antibody-drug conjugate as described herein or a pharmaceutical composition as described herein for use in the manufacture of a medicament for treatment of a disease characterised by cells expressing uPARAP, such as cancer.

In one embodiment, the present disclosure relates to an antibody, an antibody-drug conjugate or a pharmaceutical composition comprising said antibody or antibody-drug conjugate for use in the manufacture of a medicament for treatment of a disease characterised by cells expressing uPARAP, such as cancer, wherein said antibody or antibody-drug conjugate is or comprises an antibody comprising:
a. an immunoglobulin light chain variable region comprising or consisting of the amino acid sequence of SEQ ID NO: 3; and
b. an immunoglobulin heavy chain variable region comprising or consisting of the amino acid sequence of SEQ ID NO: 6.

In one embodiment, the present disclosure relates to an antibody, an antibody-drug conjugate or a pharmaceutical composition comprising said antibody or antibody-drug conjugate for use in the manufacture of a medicament for treatment of a disease characterised by cells expressing uPARAP, such as cancer, wherein said antibody or antibody-drug conjugate is or comprises an antibody comprising:
a. an immunoglobulin light chain comprising or consisting of the amino acid sequence of SEQ ID NO: 1; and
b. an immunoglobulin heavy chain comprising or consisting of the amino acid sequence of SEQ ID NO: 4.

In one embodiment, the present disclosure relates to an antibody, an antibody-drug conjugate or a pharmaceutical composition comprising said antibody or antibody-drug conjugate for use in the manufacture of a medicament for treatment of a disease characterised by cells expressing uPARAP, such as cancer, wherein said antibody or antibody-drug conjugate is or comprises an antibody comprising:
a. an immunoglobulin light chain consisting of the amino acid sequence of SEQ ID NO: 1; and
b. an immunoglobulin heavy chain consisting of the amino acid sequence of SEQ ID NO: 4.

### EXAMPLES

### Example 1: Humanization of Murine 9b7 Antibody and Potency of ADCs Based Thereon

### Materials and Methods

Humanization of the Murine Antibody 9b7 Directed Against uPARAP

Data on the murine antibody 9b7 amino acid sequence and its CDR regions are available in the published patent application WO 2017/133745.

Humanized variants of the 9b7 antibody were constructed by a third party (Fusion Antibodies, Belfast, UK). Briefly, the murine parental antibody (clone 9b7) was sequenced, and the consensus CDR sequences were grafted into human donor sequences in silico.

For this purpose, a number of human framework sequences (see search procedure below) were used as acceptor frameworks for the CDR sequences. These acceptor sequences have all come from mature Human IgG from a human source and not from phage display or other technologies. The generated humanized variants from the Antibody 9b7 sequences are combinations of light and heavy chains, referred to as Ab 980.2 LCXHCX (Light chain X, Heavy chain X), except that LC0HC0 refers to the chimeric antibody in which the variable domains of the original murine antibody is fused to the same human IgG constant regions as used in the humanized antibodies. The mature humanized antibodies are complete IgG molecules of the IgG1 Kappa type.

For the heavy chain, online databases of Human IgG sequences were searched for comparison to the murine VH domain using BLAST search algorithms, and candidate human variable domains selected from the top 200 BLAST results. These were reduced to four candidates based on a combination of framework homology, maintaining key framework residues and canonical loop structure.

For the light chain, online databases of Human IgK sequences were searched for comparison to the murine VL domain using BLAST search algorithms, and candidate human variable domains selected from the top 200 BLAST results. These were reduced to four candidates based on a combination of framework homology, maintaining key framework residues and canonical loop structure.

Altogether, DNA sequences encoding 4 humanized light chains and 4 humanized heavy chains were thus selected. All of the resulting 16 light- and heavy chain combinations were used for protein expression in CHO cells. To enable protein expression, each of the Variable Light Chain domains was positioned in-frame with a human IgK isotype constant domain sequence, while each of the Variable Heavy Chain domains was positioned in-frame with a human IgG1 isotype constant domain sequence. The chimeric antibody, LC0HC0, in which the variable domains of the murine protein were fused to the same human IgG constant regions, was expressed for comparison.

For protein expression (performed by a third party (Fusion Antibodies, Belfast, UK)), a mammalian expression vector encoding each variant was transfected into CHO cells and batch cultures of each variant grown for up to seven days. The expressed antibodies were then subsequently purified from cell culture supernatant via affinity chromatography. The concentration and purity were determined for the purified antibody products.

The obtained sequences were cloned into the mammalian transient expression plasmid pD2610-v13 (ATUM). The humanized antibody variants were expressed using a CHO based transient expression system and the resulting antibody containing cell culture supernatants were clarified by centrifugation and filtration. The humanized variants were then purified (using state-of-the-art AKTA chromatography equipment) from cell culture supernatants via affinity chromatography. Purified antibodies were dialysed/buffer exchanged into phosphate buffered saline solution. The purity of the antibody was determined to be >95%, as judged by Sodium Dodecyl Sulphate Polyacrylamide gels Among the resulting 16 humanized antibodies, the combination designated LC4HC3 was selected for further study based on favorable protein expression yields and antigen binding properties. Another humanized antibody designated LC3HC3 was selected for comparison with LC4HC3 of key parameters such as manufacturability, internalization and in vivo efficacy.

### Spr Analyses for Determination of Antibody-Ligand Affinity

Once suitable antibodies are obtained, they may be tested for antigen specificity, for example by surface plasmon resonance (SPR) or ELISA. When a soluble recombinant protein consisting of the three N-terminal domains of uPARAP (CysR, FN-II and CTLD-1) is immobilized in a BIAcore setup, mAb 9b7 binds to this construct.

SPR analyses were performed for determination of the affinity of the obtained antibodies towards uPARAP. These analyses were performed using a Biacore 2000 instrument (Biaffin GmbH, Kassel, Germany) using a CM5 sensor chip with an anti-human Fc capture surface for antibody binding. The analysis temperature was set at 25° C. Binding of antibody onto this surface was followed by passing soluble full-length uPARAP over the chip, and the resulting association and dissociation rates were derived from the resulting binding curves. For kinetic interaction analyses, a flowrate of 30 μL/min was used and the analysis buffer consisted of 10 mM HEPES pH 7.4, 150 mM NaCl, 3 mM EDTA, 0.05% Tween 20.

### Preparation and Evaluation of Antibody-Drug Conjugates (ADCs)

ADCs used for these studies were generated using a well-established conjugation approach. In brief, targeting antibodies were subjected to conjugation to a “vedotin” type of payload (MC-VC-PABC-MMAE) by mild reduction of interchain disulphides, followed by conjugation to a surplus of the payload via the maleimide group to a moderate average drug-to-antibody ratio (DAR) of around 4. ADCs were then purified using PD-10 desalting columns (GE Healthcare).

### Cell Lines

The U937 cell line was obtained from ATCC, and maintained in RPMI, 10% fetal bovine serum, 1% penicillin/streptomycin, in a 37 degrees Celsius incubator in a 5% CO2 atmosphere.

### In Vitro Cytotoxicity of ADCs—Cell Viability Assay

U937 cells were seeded at low density (20% confluence, 2×103 cells per well) in a flat-bottom 96 well plate in 90 μL of medium and incubated overnight. The next day, MMAE-based ADCs of the LC4HC3 and LC0HC0 antibodies, comparably synthesized using the method described above, were prepared as a serial dilution (1:4) in PBS and added in volumes of 10 μL to each well, with a final maximum ADC concentration of 0.1 μg/mL ADC (mAb component). Cells were incubated for 96 hours, before 12 μL of CellTiter 96 AQueous One Solution Cell Proliferation Assay (MTS, Promega) was added, and incubated for an appropriate time for formation of color (around 60 minutes). The plates were then read at 490 nm, with background subtraction at 630 nm, using a plate reader, to yield the resulting viability estimates. Cells treated with PBS only were used as a nontreated control, the viability of which the ADC treated wells were normalized to.

### Results

Manufacturability and expression of LC4HC3 and LC3HC3: The humanized antibodies designated as LC4HC3 and LC3HC3 were expressed in CHO cells and purified as described above. The same procedure was implemented for both antibodies. Results are summarized below in Table 1, clearly showing that the LC4HC3 can be produced in significantly higher quantities at sufficient purity:

TABLE 1
manufacturability data for humanized
antibodies LC3HC3 and LC4HC3

| Antibody | Conc. | Vol. | Total | Estimated |
|---|---|---|---|---|
| ID | (mg/ml) | (ml) | (mg) | purity (%) |
| LC4HC3 | 3.69 | 3.02 | 11.14 | >95% |
| LC3HC3 | 2.42 | 3.06 | 7.41 | >95% |

### Spr Analyses:

LC4HC3 and LC0HC0 were analyzed by SPR as described in Materials and Methods above. In particular, the binding kinetics of LC4HC3 were compared with those of LC0HC0 (Table 2). As evident from these analyses, the lower KDfor the antibody LC4HC3 indicates an approx. 1.7-fold higher ligand affinity than the parental antibody, LC0HC0.

TABLE 2
result overview from kinetic interaction
analysis based on SPR measurements

| | Kinetic global fit (Langmuir 1:1) | | | | |
|---|---|---|---|---|---|
| | | | | | % |
| Measure- | kₐ | k_{d} | | | active |
| ment | (M⁻¹s⁻¹) | (s⁻¹) | K_{D} (M) | Rₘₐₓ | ligand |
| LC0HC0 | 6.23 × 10⁴ | 5.33 × 10⁻⁵ | 8.6 × 10⁻¹⁰ | 35.9 RU | 81% |
| LC4HC3 | 8.13 × 10⁴ | 4.04 × 10⁻⁵ | 5.0 × 10⁻¹⁰ | 36.6 RU | 123% |

### ADC In Vitro Potency Analysis:

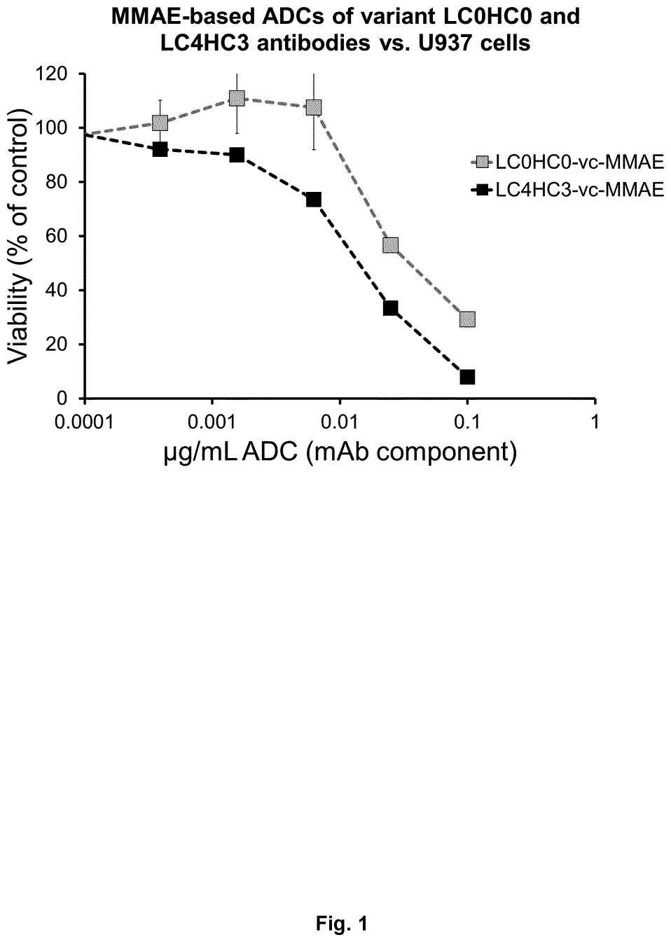

MMAE-containing ADCs including either of the antibodies LC4HC3 and LC0HC0 were prepared as described above. The in vitro cytotoxicity of these ADCs was tested against uPARAP-positive U937 cells using concentration series of the ADCs (FIG. 1). It is evident that the amount of ADC needed for cell eradication is lower for the LC4HC3-based than for the LC0HC0-based ADC, as the viability curves resulting from treatment with LC4HC3-vc-MMAE are shifted several fold towards lower concentration, compared to the curve resulting from treatment with LC0HC0-vc-MMAE.

### Conclusion

A humanized antibody, 980.2 LC4HC3, has been developed from the murine monoclonal antibody, mAb 9b7. The properties of this novel antibody can be compared directly with those resulting from the parental variable sequences by comparison with the chimeric antibody, 980.2 LC0HC0, in which the entire murine variable sequences are retained in an otherwise human IgG setting. This comparison reveals that, 1) humanized 980.2 LC4HC3 has a higher ligand affinity than 980.2 LC0HC0, and 2) an ADC based on 980.2 LC4HC3 is more efficient in terms of cytotoxicity than an otherwise equivalent ADC based on 980.2 LC0HC0.

### Example 2: Internalization of Humanized Variants of Murine 9b7 Antibody, LC3HC3 and LC4HC3

### Materials and Methods

### Antibody Labeling

Iodogen (Thermo Fischer) was dissolved at 120 μg/ml in chloroform, and used to coat the bottom of glass tubes by evaporation. In coated tubes, 200 μg/ml of humanized antibody (either LC3HC3 or LC4HC3) reacted with 588 ng/ml 1-125 (Perkin Elmer) in a 0.1 M TRIS buffer at pH 7.6 for 10 minutes. The reaction was stopped by addition of a nine-fold excess of 0.1 M Tris pH 8.1 buffer containing 0.01% Tween-80. Non-bound iodine was separated from collagen over a PD-10 column, labeled antibody was eluted in a 0.1 M Tris/HCl buffer with pH 8.1, 0.01% Tween-80. Assuming all antibody is eluted in this buffer leads to a concentration of 8 μg/ml. The integrity and radioactivity of the labeled collagen was routinely confirmed by running it on SDS-PAGE, followed by Coomassie staining and phosphorimaging.

### Cell Culture and Antibody Internalization Procedure

SAOS-2 osteosarcoma cells (Finsenlab; viability 98.7%, density of 1.07×10\hat{ }6/ml) were diluted to 1×10\hat{ }5/ml and 1 ml per 24-well was seeded for experiments. Cells were allowed to adhere overnight. At least 30 minutes prior to the addition of radiolabeled antibodies the medium was replaced by internalization medium, consisting of DMEM/F12 with 1,5% FBS and 20 mM HEPES. Internalization medium without cells was seeded in separate wells as controls. It is assumed that radioactivity from these samples represents the amount of radiolabeled protein that sticks to plastic and is retrieved upon trypsin treatment. These measurements could be considered “baseline levels” and could be subtracted from measurements in samples that did contain cells. 5 μl of LC4HC3 or LC3HC3, presumed to be slightly less than 40 ng based on assumptions mentioned above, was added to each well. After 1 hour or 4 hours, media was removed by suction and the cells were washed three times with 500 μl ice cold PBS. 500 μl of Trypsin-EDTA with 50 μg/ml proteinase K was added to each well for 2 minutes. Cells were harvested, transferred to Eppendorf tubes and spun at 1000 g, 4° C., for 1 minute. Supernatants (containing cell-bound antibodies) and pellets were collected separately and analyzed on a gamma counter. 2 μl of labeled antibody stock was analyzed simultaneously to assess labeling efficiency.

### Results

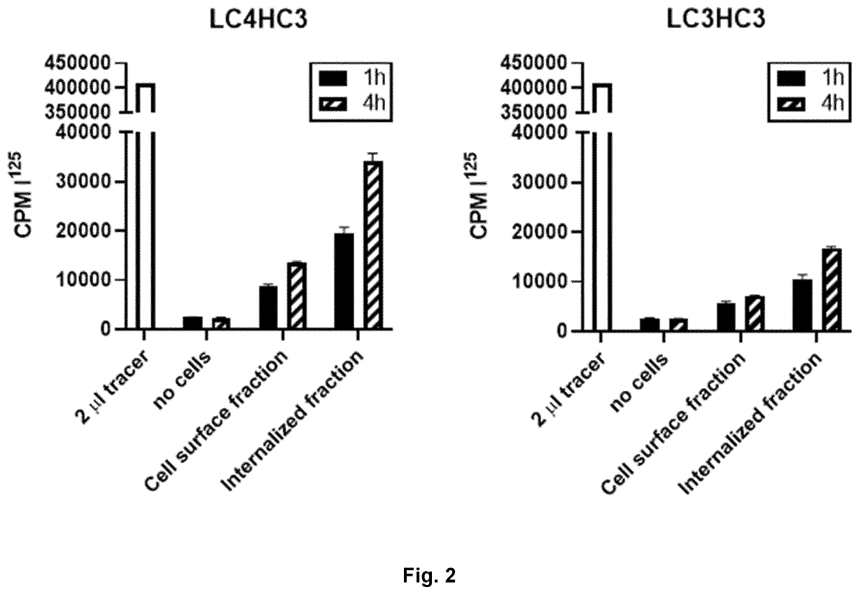

The results presented in FIG. 2 clearly illustrate that the humanized antibody LC4HC3 is internalized not only significantly faster, but also to a larger extent than the humanized antibody LC3HC3.

### Conclusion

Humanized antibody LC4HC3 was internalized to the largest extent by SAOS-2 osteosarcoma cells, in a time-dependent manner. LC3HC3 was also internalized, but to a far less extent, and not as quickly as LC4HC3. The two referenced antibodies comprise the same heavy chain, and the difference in internalization can be attributed solely to the difference in amino acid sequence of the light chain.

### Example 3: In Vivo Efficacy of ADCs Based on Humanized Murine 9b7 Antibodies LC3HC3 and LC4HC3

### Materials and Methods

### Cell Culture and Preparation

U937 cells (as described above) were passaged according to standard procedures until enough cells for this experiment were acquired. Cells were spun down at 150 g for 5 min and washed 3 times in cold PBS (Gibco). The cell concentration was adjusted to 3.6×10⁶ cells/ml. This translates to approximately 3 million live cells per 100 μl, which is the intended inoculation volume.

### Xenograft Tumor Inoculation

Recipient CB17 mice were anesthetized with Zoletil (AEM), Viscotears eye drops were applied, and earmarks were made. The right flank was shaved and disinfected with 70% ethanol. A 25 G needle was used to inject 100 μl of re-suspended U937 cells into the subcutaneous space (no incision or suture necessary). The mice were allowed to recover from the anesthesia in their cages. Recovery was monitored until the mice were mobile. The mice were monitored again the next day, and tumor sizes were monitored closely until the start of treatment.

Treatments were initiated as soon as the tumors reached a proper size (approximately 80-150 mm³).

### ADC Treatment and Monitoring

Vedotin-type (MMAE) ADCs comprising the LC3HC3 or LC4HC3 humanized antibodies were prepared as previously described above. The mice were divided into groups, with 3-5 animals per group (N=3-5), the groups differing by the employed ADC or dose administered of said ADC. One cohort of mice was tested with the LC3HC3 ADC in groups receiving concentrations of 2, 4 or 6 mg/kg and another cohort was tested with the LC4HC3 ADC in groups receiving the same range of concentrations.

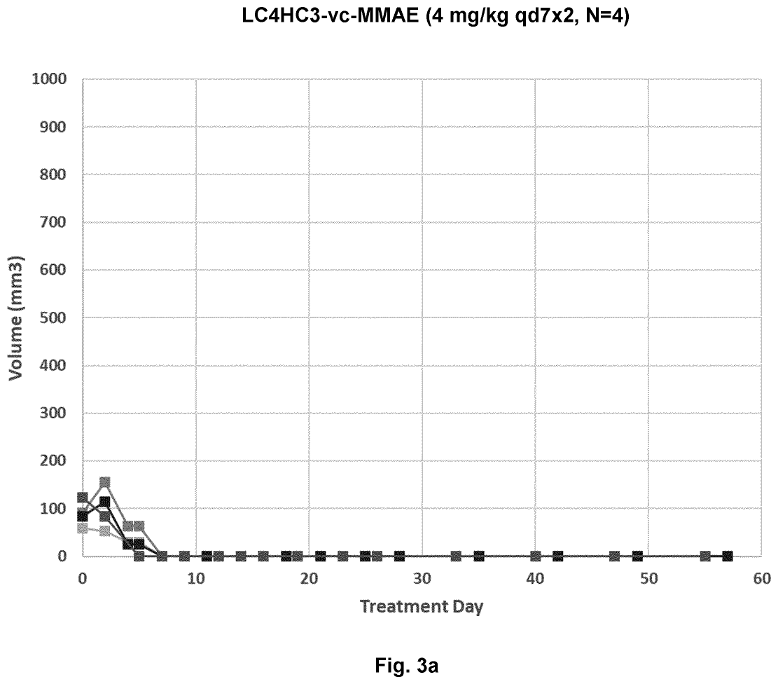

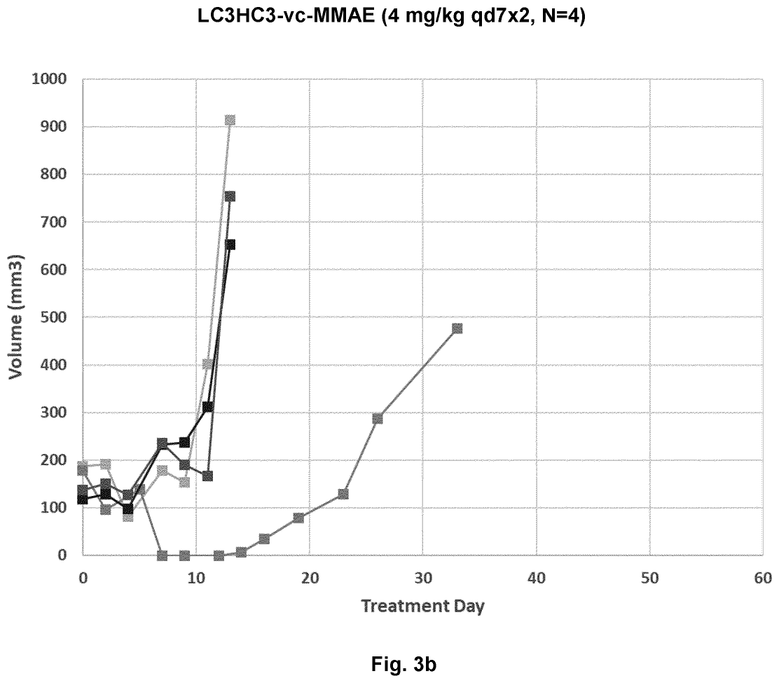

For each group, mice were administered a controlled amount of ADC intravenously (tail vein) once weekly, for two injections total (qd7×2), and tumor progression was closely monitored also post-treatment. Exemplary, FIGS. 3a and 3b show the tumor progression in groups treated with a 4 mg/kg dose of LC4HC3 and LC3HC3 ADCs respectively.

The monitoring consists of checking for overall wellbeing, and measuring the width and length dimensions of the tumor with a digital caliper. All observations and measurements are noted by hand and transferred to the digital data sheets following inspection. Animals were euthanized if the tumor size exceeded 12 mm in one dimension, if the volume of the tumor (calculated as (length×width²)/2) exceeded 1000 mm³, or if a severe impact on general wellbeing was observed. Animals were euthanized via cervical dislocation.

### Results

U937 tumor volumes following treatment with ADCs based on LC4HC3 and LC3HC3 are found in FIGS. 3a and 3b respectively. FIG. 3a shows tumor volumes for a group of four mice (N=4), each administered the LC4HC3 ADC in a 4 mg/kg once weekly for two injections total (qd7×2), and FIG. 3b shows tumor volumes for a different group of four mice (N=4), each administered the LC3HC3 ADC in a 4 mg/kg dose once weekly for two injections total (qd7×2).

### Conclusion

As evident when comparing FIGS. 3a and 3b, the treatment based on LC4HC3 ADCs completely cured all mice with no regrowth of tumor in the post-treatment period, notably in all doses tested. In contrast, the same treatment based on LC3HC3 was not able to kill all tumor cells, and in instances, aggressive tumor growth was observed in the post-treatment monitoring period.

The data shows that humanized antibody LC4HC3 as well as ADCs comprising said humanized antibody are potent antitumor agents with improved in vivo efficacy compared to another humanized antibody, LC3HC3. The two referenced antibodies comprise the same heavy chain, and the difference in efficacy can be attributed solely to the variations in amino acid sequence of the light chain.

Sequence overview

| SEQ ID | | |
|---|---|---|
| NO | Sequence | Comment |
| 1 | EIVMTQSPDSLAVSLGERATINCKASQNVDTYVVWYQQ | Complete |
| | KPGQPPQPLIYSASSRFSGVPDRFSGSGSGTDFTLTISS | light chain |
| | LQAEDVAIYYCQQYHNSPLTFGGGTKVEIKRTVAAPSVFI | sequence |
| | FPPSDEQLKSGTASVVCLLNNFYPREAKVQWKVDNALQ | (LC4) of |
| | SGNSQESVTEQDSKDSTYSLSSTLTLSKADYEKHKVYA | humanised |
| | CEVTHQGLSSPVTKSFNRGEC | LC4HC3 |
| 2 | MVSSAQFLGLLLLCFQGTRCEIVMTQSPDSLAVSLGERA | Complete |
| | TINCKASQNVDTYVVWYQQKPGQPPQPLIYSASSRFSG | light chain |
| | VPDRFSGSGSGTDFTLTISSLQAEDVAIYYCQQYHNSPL | sequence |
| | TFGGGTKVEIKRTVAAPSVFIFPPSDEQLKSGTASVVCLL | (LC4) of |
| | NNFYPREAKVQWKVDNALQSGNSQESVTEQDSKDSTY | humanised |
| | SLSSTLTLSKADYEKHKVYACEVTHQGLSSPVTKSFNRG | LC4HC3 in |
| | EC | combination |
| | | with signal |
| | | peptide |
| | | used in |
| | | CHO cell |
| | | expression |
| | | system; |
| | | Signal |
| | | peptide |
| | | underlined |
| 3 | EIVMTQSPDSLAVSLGERATINCKASQNVDTYVVWYQQ | Light chain |
| | KPGQPPQPLIYSASSRFSGVPDRFSGSGSGTDFTLTISS | (LC4) |
| | LQAEDVAIYYCQQYHNSPLTFGGGTKVEIK | variable |
| | | region of |
| | | humanised |
| | | LC4HC3 |
| 4 | QVQLVQSGAEVKKPGASVKVSCKASGYIFIDYGMHWVR | Complete |
| | QAPGQRLEWMGSINTKSGVSTYAAEFKGRVTIYSDTSA | heavy chain |
| | STAYMELSSLRSEDTAVYFCARPPYYSQYGSYWGQGTL | sequence |
| | VTVSSASTKGPSVFPLAPSSKSTSGGTAALGCLVKDYFP | (HC3) of |
| | EPVTVSWNSGALTSGVHTFPAVLQSSGLYSLSSVVTVP | humanised |
| | SSSLGTQTYICNVNHKPSNTKVDKKVEPKSCDKTHTCPP | LC4HC3 |
| | CPAPELLGGPSVFLFPPKPKDTLMISRTPEVTCVVVDVS | and |
| | HEDPEVKFNWYVDGVEVHNAKTKPREEQYNSTYRVVS | LC3HC3 |
| | VLTVLHQDWLNGKEYKCKVSNKALPAPIEKTISKAKGQP | |
| | REPQVYTLPPSRDELTKNQVSLTCLVKGFYPSDIAVEWE | |
| | SNGQPENNYKTTPPVLDSDGSFFLYSKLTVDKSRWQQG | |
| | NVFSCSVMHEALHNHYTQKSLSLSPGK | |
| 5 | MGWTLVFLFLLSVTAGVHSQVQLVQSGAEVKKPGASVK | Complete |
| | VSCKASGYIFIDYGMHWVRQAPGQRLEWMGSINTKSGV | heavy chain |
| | STYAAEFKGRVTIYSDTSASTAYMELSSLRSEDTAVYFC | sequence |
| | ARPPYYSQYGSYWGQGTLVTVSSASTKGPSVFPLAPSS | (HC3) of |
| | KSTSGGTAALGCLVKDYFPEPVTVSWNSGALTSGVHTF | humanised |
| | PAVLQSSGLYSLSSVVTVPSSSLGTQTYICNVNHKPSNT | LC4HC3 |
| | KVDKKVEPKSCDKTHTCPPCPAPELLGGPSVFLFPPKPK | and |
| | DTLMISRTPEVTCVVVDVSHEDPEVKFNWYVDGVEVHN | LC3HC3 in |
| | AKTKPREEQYNSTYRVVSVLTVLHQDWLNGKEYKCKVS | combination |
| | NKALPAPIEKTISKAKGQPREPQVYTLPPSRDELTKNQV | with signal |
| | SLTCLVKGFYPSDIAVEWESNGQPENNYKTTPPVLDSD | peptide |
| | GSFFLYSKLTVDKSRWQQGNVF | used in |
| | SCSVMHEALHNHYTQKSLSLSPGK | CHO cell |
| | | expression |
| | | system; |
| | | Signal |
| | | peptide |
| | | underlined |
| 6 | QVQLVQSGAEVKKPGASVKVSCKASGYIFIDYGMHWVR | Heavy |
| | QAPGQRLEWMGSINTKSGVSTYAAEFKGRVTIYSDTSA | chain (HC3) |
| | STAYMELSSLRSEDTAVYFCARPPYYSQYGSYWGQGTL | variable |
| | VTVSS | region of |
| | | humanised |
| | | LC4HC3 |
| | | and |
| | | LC3HC3 |
| 7 | DIVMTQSQKFMSTSVGDRVSVTCKASQNVDTYVVWYQ | Complete |
| | QKPGQSPKPLIYSASSRFSGVPDRFTGTGSGTDFTLTIN | light chain |
| | NVQSEDLAEYFCQQYHNSPLTFGGGTKLEIKRTVAAPSV | sequence |
| | FIFPPSDEQLKSGTASVVCLLNNFYPREAKVQWKVDNAL | (LC0) of |
| | QSGNSQESVTEQDSKDSTYSLSSTLTLSKADYEKHKVY | chimeric |
| | ACEVTHQGLSSPVTKSFNRGEC | LC0HC0 |
| 80 | DIVMTQSQKFMSTSVGDRVSVTCKASQNVDTYVVWYQ | Light chain |
| | QKPGQSPKPLIYSASSRFSGVPDRFTGTGSGTDFTLTIN | (LC0) |
| | NVQSEDLAEYFCQQYHNSPLTFGGGTKLEIK | variable |
| | | region of |
| | | chimeric |
| | | LC0HC0 |
| 9 | QVHLVQSGPELKKPGETVKISCKASGYIFIDYGMHWVKQ | Complete |
| | APGKGLKWMGSINTKSGVSTYAAEFKGRFAFSLETSAS | heavy chain |
| | TAYLQINNLKNEDTATYFCARPPYYSQYGSYWGQGTLV | sequence |
| | TVSAASTKGPSVFPLAPSSKSTSGGTAALGCLVKDYFPE | (HC0) of |
| | PVTVSWNSGALTSGVHTFPAVLQSSGLYSLSSVVTVPS | chimeric |
| | SSLGTQTYICNVNHKPSNTKVDKKVEPKSCDKTHTCPPC | LC0HC0 |
| | PAPELLGGPSVFLFPPKPKDTLMISRTPEVTCVVVDVSH | |
| | EDPEVKFNWYVDGVEVHNAKTKPREEQYNSTYRVVSVL | |
| | TVLHQDWLNGKEYKCKVSNKALPAPIEKTISKAKGQPRE | |
| | PQVYTLPPSRDELTKNQVSLTCLVKGFYPSDIAVEWESN | |
| | GQPENNYKTTPPVLDSDGSFFLYSKLTVDKSRWQQGNV | |
| | FSCSVMHEALHNHYTQKSLSLSPGK | |
| 10 | QVHLVQSGPELKKPGETVKISCKASGYIFIDYGMHWVKQ | Heavy |
| | APGKGLKWMGSINTKSGVSTYAAEFKGRFAFSLETSAS | chain (HC0) |
| | TAYLQINNLKNEDTATYFCARPPYYSQYGSYWGQGTLV | variable |
| | TVSA | region of |
| | | chimeric |
| | | LC0HC0 |
| 11 | ATGGTCAGCTCTGCTCAATTTCTCGGACTCCTTCTTCT | Polynucleotide |
| | GTGCTTTCAAGGAACACGCTGCGAGATCGTGATGACT | encoding |
| | CAGTCCCCGGACTCACTGGCAGTGTCCTTGGGCGAA | LC4 incl. |
| | AGAGCCACCATCAACTGTAAAGCCAGCCAGAACGTGG | signal |
| | ACACCTACGTGGTCTGGTACCAGCAGAAGCCTGGACA | peptide |
| | GCCACCGCAGCCGTTGATCTACTCGGCCTCATCAAGG | |
| | TTCTCCGGGGTGCCGGACCGCTTCTCCGGATCCGGC | |
| | TCCGGCACCGATTTCACCCTGACCATCTCCTCACTGC | |
| | AAGCCGAGGACGTGGCTATCTACTATTGCCAGCAGTA | |
| | CCACAACTCCCCACTGACCTTCGGTGGCGGAACTAAG | |
| | GTCGAGATTAAGCGGACCGTGGCGGCCCCCTCTGTG | |
| | TTCATTTTCCCTCCCTCGGACGAACAGCTGAAGTCGG | |
| | GAACAGCCTCCGTCGTGTGCCTGCTCAACAACTTCTA | |
| | CCCCCGGGAAGCGAAGGTCCAGTGGAAAGTGGATAA | |
| | CGCACTCCAATCGGGGAACTCCCAGGAATCCGTGACT | |
| | GAGCAGGACTCGAAGGATTCCACTTACTCCCTGTCGT | |
| | CCACCCTGACTCTGAGCAAGGCCGACTACGAGAAGC | |
| | ATAAGGTCTACGCCTGCGAAGTGACCCACCAGGGTCT | |
| | GAGCTCCCCTGTGACCAAGAGCTTTAATCGGGGCGAA | |
| | TGTTGA | |
| 12 | ATGGGTTGGACCCTCGTCTTTCTGTTCCTTCTTTCCGT | Polynucleotide |
| | CACCGCTGGAGTGCATAGCCAGGTCCAATTGGTGCA | encoding |
| | GTCAGGCGCCGAAGTGAAAAAGCCTGGGGCGTCGGT | HC3 incl. |
| | GAAAGTGTCCTGCAAAGCCTCGGGCTACATCTTTATT | signal |
| | GACTACGGAATGCACTGGGTCCGCCAGGCCCCGGGC | peptide |
| | CAGAGGCTGGAGTGGATGGGATCCATTAACACCAAGA | |
| | GCGGAGTGTCAACTTACGCAGCCGAGTTCAAGGGAC | |
| | GGGTGACCATCTATAGCGATACCTCTGCGTCGACCGC | |
| | CTACATGGAATTGTCATCACTCCGGTCCGAGGACACT | |
| | GCCGTGTACTTCTGCGCAAGGCCACCCTACTACTCGC | |
| | AATACGGCAGCTACTGGGGCCAGGGAACACTTGTGA | |
| | CCGTGTCGAGCGCGTCCACCAAGGGTCCCTCCGTGT | |
| | TCCCTCTCGCGCCGTCCTCAAAGTCTACCTCCGGTGG | |
| | AACTGCCGCGCTCGGTTGTCTCGTGAAGGACTACTTC | |
| | CCGGAGCCTGTGACTGTCTCCTGGAACTCCGGGGCC | |
| | CTCACCAGCGGAGTGCACACTTTCCCCGCCGTGCTG | |
| | CAATCCTCCGGCCTGTACAGCCTGTCCTCCGTCGTGA | |
| | CTGTGCCTAGCTCCTCCCTGGGAACCCAGACCTACAT | |
| | CTGCAACGTGAACCACAAGCCCTCCAACACCAAGGTC | |
| | GACAAGAAGGTCGAACCGAAGTCGTGCGACAAGACT | |
| | CATACGTGCCCTCCTTGCCCGGCCCCGGAACTGCTG | |
| | GGAGGCCCATCCGTGTTCCTGTTCCCACCCAAGCCTA | |
| | AGGATACCCTGATGATCAGCAGAACACCGGAAGTGAC | |
| | CTGTGTGGTGGTGGACGTCAGCCACGAAGATCCCGA | |
| | GGTCAAGTTCAATTGGTACGTGGACGGGGTGGAGGT | |
| | GCACAACGCAAAGACCAAGCCCCGGGAGGAACAGTA | |
| | CAACTCCACCTATCGCGTGGTGTCGGTGCTGACGGT | |
| | GCTGCACCAGGACTGGTTGAACGGAAAGGAGTATAA | |
| | GTGCAAAGTGTCGAACAAGGCCCTGCCCGCTCCTATC | |
| | GAAAAGACCATCTCCAAGGCCAAGGGCCAGCCGCGG | |
| | GAACCCCAGGTCTACACTCTCCCACCGAGCCGCGAC | |
| | GAACTGACTAAGAATCAAGTGTCGCTGACTTGCCTCG | |
| | TCAAGGGCTTCTACCCGTCCGACATCGCCGTGGAATG | |
| | GGAGAGCAACGGCCAGCCGGAAAACAACTACAAGAC | |
| | CACCCCTCCCGTGCTGGATTCCGACGGGTCCTTCTTC | |
| | CTGTACTCAAAACTGACCGTGGATAAGTCCAGATGGC | |
| | AGCAGGGCAATGTCTTTTCATGCTCCGTGATGCACGA | |
| | GGCTCTGCATAACCACTACACCCAGAAGTCGCTGTCC | |
| | CTGTCCCCGGGGAAGTGA | |
| 13 | MVSSAQFLGLLLLCFQGTRCDWVMTQSPDSLAVSLGER | Complete |
| | VTINCKASQNVDTYWWYQQKPGQSPKLLIYSASSRFSG | light chain |
| | VPDRFSGSGSGTDFTLTISSLQAEDVAVYYCQQYHNSPL | sequence |
| | TFGQGTKLEIKRTVAAPSVFIFPPSDEQLKSGTASVVCLL | (LC3) of |
| | NNFYPREAKVQWKVDNALQSGNSQESVTEQDSKDSTY | humanised |
| | SLSSTLTLSKADYEKHKVYACEVTHQGLSSPVTKSFNRG | LC3HC3 |
| | EC | |

### Items 1

1. An antibody which binds to uPARAP comprising:
a. an immunoglobulin light chain variable region comprising or consisting of the amino acid sequence of SEQ ID NO: 3; and/or
b. an immunoglobulin heavy chain variable region comprising or consisting of the amino acid sequence of SEQ ID NO: 6.

2. The antibody according to item 1, wherein said antibody comprises:
a. an immunoglobulin light chain comprising the amino acid sequence of SEQ ID NO: 1; and/or
b. an immunoglobulin heavy chain comprising the amino acid sequence of SEQ ID NO: 4.

3. The antibody according to any one of the preceding items, wherein said antibody comprises:
a. an immunoglobulin light chain consisting of the amino acid sequence of SEQ ID NO: 1; and
b. an immunoglobulin heavy chain consisting of the amino acid sequence of SEQ ID NO: 4.

4. An antibody-drug conjugate (ADC) comprising:
a. the antibody according to any one of the preceding items,
b. an active agent, and
c. optionally a linker which links a) to b).

5. The antibody-drug conjugate according to item 4, wherein the active agent is selected from a therapeutic agent, a radioisotope, and a detectable label.

6. The antibody-drug conjugate according to any one of items 4 to 5, wherein the active agent is a cytotoxic agent.

7. The antibody-drug conjugate according to any one of items 4 to 6, wherein the active agent is a therapeutic agent, such as a therapeutic agent selected from the group consisting of anti-microtubule/anti-mitotic agents, DNA crosslinking agents, DNA alkylating agents, DNA strand scission agents, anthracyclines, antimetabolites, histone deacetylase inhibitors, kinase inhibitors, metabolism inhibitors, peptide antibiotics, immune checkpoint inhibitors, platinum-based antineoplastics, topoisomerase inhibitors, DNA or RNA polymerase inhibitors, nucleotide based agents, and cytotoxic antibiotics.

8. The antibody-drug conjugate according to any one of items 4 to 7, wherein the active agent is an anti-mitotic agent, such as selected from the group consisting of derivatives of auristatin or dolastatin such as monomethyl auristatin E (MMAE), monomethyl auristatin F (MMAF) and more, a taxane such as Paclitaxel or Docetaxel and more, a vinca alkaloid such as Vinblastine, Vincristine, Vindesine or Vinorelbine and more, a mayatansinoid, Colchicine, and Podophyllotoxin.

9. The antibody-drug conjugate according to any one of items 4 to 8, wherein the active agent is monomethyl auristatin E (MMAE).

10. The antibody-drug conjugate according to any one of items 4 to 7, wherein the active agent is a DNA-crosslinking agent, such as a DNA crosslinking agent selected from cisplatin or a derivative of cisplatin such as carboplatin or oxaliplatin, mitomycin C (MMC), pyrrolobenzodiazepine, and dimeric pyrrolobenzodiazepine derivatives such as SGD-1882.

11. The antibody-drug conjugate according to any one of items 4 to 7, wherein the active agent is a DNA alkylating agent, such as a DNA alkylating agent selected from nitrogen mustards such as tris(2-chloroethyl)amine, pyridinobenzodiazepines or a pyridinobenzodiazepine derivative, indolinobenzodiazepine dimers, and Duocarmycin SA.

12. The antibody-drug conjugate according to any one of items 4 to 7, wherein the active agent is a DNA strand scission agent, such as a DNA strand scission agent selected from calicheamicin and hamiltrone.

13. The antibody-drug conjugate according to any one of items 4 to 7, wherein the active agent is an anthracycline, such as an anthracycline selected from Daunorubicin, doxorubicin, epirubicin, idarubicin, and PNU-159682.

14. The antibody-drug conjugate according to any one of items 4 to 7, wherein the active agent is an antimetabolite, such as an antimetabolite selected from folic acid antagonists such as methotrexate, purine antimetabolites such as 6-mercaptopurine or 6-thioguanine or fludarabine phosphate or pentostatin or cladribine, and pyrimidine antimetabolites such as 5-fluorouracil or 5-fluorodeoxyuridine or cytarabine or gemcitabine.

15. The antibody-drug conjugate according to any one of items 4 to 7, wherein the active agent is a histone deacetylase inhibitor, such as a histone deacetylase inhibitor selected from trichostatin A, vorinostat, belinostat, panabiostat, givinostat, resminostat, abexinostat, quisinostat, rocilinostat, practinostat, CHR-3996, valproic acid, butyric acid, phenylbutyric acid, entinostat, tacedinaline, 4SC202, mocetinostat, romidepsin, nicotinamide, sirtinol, cambinol, and EX-527.

16. The antibody-drug conjugate according to any one of items 4 to 7, wherein the active agent is a kinase inhibitor, such as a kinase inhibitor selected from genistein, lavendustin C, PP1-AG1872, PP2-AG1879, SU6656, CGP77675, PD166285, imatinib, erlotinib, gefitinib, lavendustin A, cetuximab, UCS15A, herbimycin A, and radicicol.

17. The antibody-drug conjugate according to any one of items 4 to 7, wherein the active agent is a metabolism inhibitor, such as an NAMPT inhibitor selected from AP0866, GMX-1777, GMX-1778 ATG-019, and OT-82.

18. The antibody-drug conjugate according to any one of items 4 to 7, wherein the active agent is an immune checkpoint inhibitor, such as a PD-1 inhibitor selected from Pembrolizumab, Nivolumab, Cemiplimab, JTX-4014, Spartalizumab, Camrelizumab, Sintilimab, Tislelizumab, Toripalimab, Dostarlimab, AMP-224 and AMP-514; or a PD-L1 inhibitor selected from Atezolizumab, Avelumab, Durvalumab, KN035, CK-301, AUNP12, CA-170 and BMS-986189.

19. The antibody-drug conjugate according to any one of items 4 to 7, wherein the active agent is a platinum-based antineoplastic, such as a platinum-based antineoplastic selected from lipoplatin, cisplatin, carboplatin, oxaliplatin, nedaplatin, picoplatin, phenanthriplatin, satraplatin, and triplatin tetranitrate.

20. The antibody-drug conjugate according to any one of items 4 to 7, wherein the active agent is a topoisomerase inhibitor, such as a topoisomerase inhibitor selected from camptothecin or derivatives thereof such as topotecan, belotecan, lurtotecan, irinotecan, SN-38, exatecan, and Dxd.

21. The antibody-drug conjugate according to any one of items 4 to 7, wherein the active agent is a DNA- or RNA-polymerase inhibitor, such as a polymerase inhibitor selected from amanitin or alpha-amanitin or derivatives thereof, actinomycin D, and aphidicolin.

22. The antibody-drug conjugate according to any one of items 4 to 21, wherein the active agent comprises a radioisotope selected from 60Co, 89Sr, 90Y, 99mTc, 131I, 137Cs, 153Sm, and 223Rd.

23. The antibody-drug conjugate according to any one of items 4 to 22, wherein the drug-to-antibody ratio (DAR) is between 1 and 10, such as between 2 and 8, for example between 2 and 6, such as 2 or 4.

24. The antibody-drug conjugate according to any one of items 4 to 23, wherein the antibody-drug conjugate comprises a linker selected from a cleavable and a non-cleavable linker.

25. The antibody-drug conjugate according to any one of items 4 to 24, wherein the linker is a peptide linker.

26. The antibody-drug conjugate according to any one of items 4 to 25, wherein the linker comprises or consists of a dipeptide, such as valine-citrulline (VC) or valine-alanine (VA).

27. The antibody-drug conjugate according to any one of items 4 to 26, wherein the antibody-drug conjugate further comprises a spacer, such as a spacer comprising p-aminobenzoic acid (PAB), p-aminobenzylcarbamate (PABC), p-aminobenzoyloxycabonyl, or polyethylenglycol (PEG).

28. The antibody-drug conjugate according to any one of items 4 to 27, wherein the antibody-drug conjugate further comprises an attachment group, such as an attachment group comprising or consisting of maleimide and caproic acid (MC), N-hydroxysuccinimide, reactive attachment groups directed to modified or unmodified protein-bound carbohydrate, peptide sequences that are required for enzymatic reactions, azides or alkynes or being derived from these by reaction with the antibody or a chemically or enzymatically generated derivative thereof.

29. The antibody-drug conjugate according to any one of items 4 to 28, wherein the antibody-drug conjugate comprises or consists of:
a. the antibody as defined in item 3,
b. a VC linker,
c. an MC attachment group,
d. a PAB or a PABC spacer, and
e. MMAE as active agent.

30. The antibody-drug conjugate according to any one of items 4 to 9 and 23 to 29, wherein the antibody-drug conjugate consists of the antibody as defined in item 3 and MC-VC-PAB-MMAE.

31. The antibody-drug conjugate according to any one of items 4 to 9 and 23 to 29, wherein the antibody-drug conjugate consists of the antibody as defined in item 3 and MC-VC-PABC-MMAE.

32. A polypeptide comprising or consisting of the amino acid sequence of SEQ ID NO: 2 and/or SEQ ID NO: 5.

33. An isolated polynucleotide which encodes the amino acid sequence of any one of SEQ ID NOs: 1, 2, 3, 4, 5 and/or 6.

34. The isolated polynucleotide according to item 33, wherein the polynucleotide comprises SEQ ID NO: 11 and/or SEQ ID NO: 12.

35. A vector comprising the polynucleotide as defined in any one of items 33 to 34.

36. The vector according to item 35, wherein the vector is a mammalian expression vector.

37. The vector according to any one of items 35 to 36, wherein the vector is a plasmid vector, such as a plasmid vector selected from pD2610-v13 (ATUM), pSV and the pCMV series of plasmid vectors.

38. The vector according to any one of items 35 to 37, wherein the vector is a viral vector, such as a viral vector selected from the group consisting of adenoviral vectors, lentiviral vectors, adeno-associated viral vectors, herpesviral vectors, vaccinia viral vectors, poxviral vectors, baculoviral vectors and oncolytic viral vectors.

39. A host cell comprising the polynucleotide according to items 32 to 33 and/or the vector according to any one of items 35 to 38.

40. The host cell according to item 39, wherein the host cell is selected from the group consisting of CHO (Chinese hamster ovary) cells, COS (CV-1 (simian) in Origin, and carrying the SV40 genetic material) cells, HEK (Human embryonic kidney) cells and HeLa (Henrietta Lacks) cells.

41. The antibody according to any one of items 1 to 3 or the antibody-drug conjugate according to any one of items 4 to 31 for use as a medicament.

42. A pharmaceutical composition comprising the antibody according to any one of items 1 to 3 or the antibody-drug conjugate according to any one of items 4 to 31 and a pharmaceutically acceptable buffer, diluent, carrier, adjuvant or excipient.

43. A method for treatment of a disease characterised by cells expressing uPARAP, said method comprising administering to a subject the antibody according to any one of items 1 to 3, the antibody-drug conjugate according to any one of items 4 to 31 or the pharmaceutical composition according to item 42.

44. The method according to item 43, wherein the disease characterised by cells expressing uPARAP is selected from cancer, a bone degradation disease such as osteoporosis, fibrosis, and macrophage associated diseases or disorders such as atherosclerosis, arthritis or chronic inflammation.

45. The method according to item 44, wherein the arthritis is selected from osteoarthritis, inflammatory arthritis, rheumatoid arthritis, psoriatic arthritis, lupus, Lyme disease-induced arthritis such as Lyme arthritis, gout or pseudogout, and ankylosing spondylitis.

46. The method according to any of items 43 to 44, wherein the disease is cancer.

47. The method according to item 46, wherein the cancer is selected from sarcoma, glioblastoma, mesothelioma, colon cancer, prostate cancer, bone metastases from prostate cancer, breast cancer, head- and neck cancer and leukaemia.

48. The method according to any of items 46 to 47, wherein the cancer is a solid tumour.

49. The method according to any of items 46 to 47, wherein cancer is leukaemia, such as acute lymphoblastic leukaemia (ALL), acute myeloid leukaemia (AML), chronic lymphocytic leukaemia (CLL), and chronic myeloid leukaemia (CML).

50. The method according to any of items 46 to 48, wherein the cancer is glioblastoma.

51. The method according to any of items 46 to 48, wherein the cancer is sarcoma, such as osteosarcoma, or soft tissue sarcoma (STS).

52. The method according to item 51, wherein the soft tissue sarcoma (STS) is selected from epithelioid sarcoma, clear cell sarcoma, alveolar soft part sarcoma, extraskeletal myxoid chondrosarcoma, epithelioid hemangioendothelioma, inflammatory myofibroblastic tumor, undifferentiated embryonal sarcoma, alveolar soft part sarcoma (ASPS), angiosarcoma, chondrosarcoma, dermatofibrosarcoma protuberens (DFSP), desmoid sarcoma, Ewing's sarcoma, fibrosarcoma, myxofibrosarcome, gastrointerstinal stromal tumor (GIST), non-uterine leiomyosarcoma, uterine leiomyosarcoma, liposarcoma, malignant fibro histiocytoma (MFH), malignant peripheral nerve sheath tumor (MPNST), rhabdomyosarcoma, synovial sarcoma, and/or leiomyosarcoma (LMS).

53. The method according to any one of items 46 to 52, wherein the cancer is metastatic cancer.

54. The method according to any of items 43 to 53, wherein the antibody according to any one of items 1 to 3, the antibody-drug conjugate according to any one of items 4 to

31, or the pharmaceutical composition according to items 42 is administered parenterally, for example, intravenously, intracerebroventricularly, intraarticularly, intra-arterially, intraperitoneally, intrathecally, intraventricularly, intrasternally, intracranially, intramuscularly or subcutaneously, or by infusion techniques.

55. The method according to any of items 43 to 54, wherein the antibody according to any one of items 1 to 3, the antibody-drug conjugate according to any one of items 4 to 31, or the pharmaceutical composition according to item 42 is administered intravenously.

56. The method according to any of items 43 to 55, wherein the antibody according to any one of items 1 to 3, the antibody-drug conjugate according to any one of items 4 to 31, or the pharmaceutical composition according to item 42 is administered in combination with one or more further agents, such as one or more further therapeutic agents.

57. The method according to any of items 43 to 56, wherein the cells expressing uPARAP display uPARAP overexpression.

58. The method according to any of items 43 to 57, wherein the cells expressing uPARAP are tumour cells and/or tumour associated cells.

59. The method according to any of items 43 to 58, wherein the antibody according to any one of items 1 to 3, the antibody-drug conjugate according to any one of items 4 to 31, or the pharmaceutical composition according to item 42 induces cell death and/or inhibits the growth and/or proliferation of the uPARAP expressing cells.

60. The method according to any of items 43 to 59, wherein the antibody according to any one of items 1 to 3, the antibody-drug conjugate according to any one of items 4 to 31, or the pharmaceutical composition according to item 42 induces liberation of free cytotoxin from the uPARAP expressing cells, leading to cell death and/or inhibition of the growth and/or proliferation of neighbouring cancer cells.

61. The method according to any of items 43 to 60, wherein the treatment is ameliorative or curative.

62. A method for inhibiting tumour progression in a subject, comprising administering to the subject the antibody according to any one of items 1 to 3, the antibody-drug conjugate according to any one of items 4 to 31, or the pharmaceutical composition according to item 42.

63. A method for inhibiting, lowering or eliminating metastatic capacity of an uPARAP expressing tumour in a subject, comprising administering to the subject the antibody according to any one of items 1 to 3, the antibody-drug conjugate according to any one of items 4 to 31, or the pharmaceutical composition according to item 42.

64. A kit comprising the antibody according to any one of items 1 to 3, the antibody-drug conjugate according to any one of items 4 to 31, or the pharmaceutical composition according to item 42, optionally further comprising means for administering the antibody or antibody-drug conjugate to a subject and/or instructions for use.

65. The antibody according to any one of items 1 to 3, the antibody-drug conjugate according to any one of items 4 to 31, or the pharmaceutical composition according to item 42 for use in the manufacture of a medicament for treatment of a disease characterised by cells expressing uPARAP, such as cancer.

### Items 2

1. An antibody which binds to uPARAP comprising:
   a. an immunoglobulin light chain variable region comprising or consisting of the amino acid sequence of SEQ ID NO: 3; and/or
   b. an immunoglobulin heavy chain variable region comprising or consisting of the amino acid sequence of SEQ ID NO: 6.
2. An antibody-drug conjugate (ADC) comprising:
   a. the antibody according to item 1,
   b. an active agent, and
   c. optionally a linker which links a) to b).
3. The antibody-drug conjugate according to item 2, wherein the active agent is a therapeutic agent, such as a therapeutic agent selected from the group consisting of anti-microtubule/anti-mitotic agents, DNA crosslinking agents, DNA alkylating agents, DNA strand scission agents, anthracyclines, antimetabolites, histone deacetylase inhibitors, kinase inhibitors, metabolism inhibitors, peptide antibiotics, immune checkpoint inhibitors, platinum-based antineoplastics, topoisomerase inhibitors, DNA or RNA polymerase inhibitors, nucleotide based agents, and cytotoxic antibiotics.
4. The antibody-drug conjugate according to any one of items 2 to 3, wherein the antibody-drug conjugate comprises a linker selected from a cleavable and a non-cleavable linker.
5. The antibody-drug conjugate according to any one of items 2 to 4, wherein the antibody-drug conjugate further comprises a spacer, such as a spacer comprising p-aminobenzoic acid (PAB), p-aminobenzylcarbamate (PABC), p-aminobenzoyloxycabonyl, or polyethylenglycol (PEG).
6. The antibody-drug conjugate according to any one of items 2 to 5, wherein the antibody-drug conjugate comprises or consists of:
   a. the antibody as defined in item 1,
   b. a VC linker,
   c. an MC attachment group,
   d. a PAB or a PABC spacer, and
   e. MMAE as active agent.
7. A polypeptide comprising or consisting of the amino acid sequence of SEQ ID NO: 2 and/or SEQ ID NO: 5.
8. An isolated polynucleotide which encodes the amino acid sequence of any one of SEQ ID NOs: 1, 2, 3, 4, 5 and/or 6.
9. The antibody according to item 1, or the antibody-drug conjugate according to any one of items 2 to 6 for use as a medicament.
10. A pharmaceutical composition comprising the antibody according to item 1, or the antibody-drug conjugate according to any one of items 2 to 6 and a pharmaceutically acceptable buffer, diluent, carrier, adjuvant or excipient.
11. The antibody according to item 1, the antibody-drug conjugate according to any one of items 2 to 6, or the pharmaceutical composition according to item 10, for use in a method of treatment of a disease characterised by cells expressing uPARAP.
12. The antibody according to item 1, the antibody-drug conjugate according to any one of items 2 to 6, or the pharmaceutical composition according to item 10 for use according to item 11, wherein the disease characterised by cells expressing uPARAP is selected from cancer, a bone degradation disease such as osteoporosis, fibrosis, and macrophage associated diseases or disorders such as atherosclerosis, arthritis or chronic inflammation.
13. The antibody according to item 1, the antibody-drug conjugate according to any one of items 2 to 6, or the pharmaceutical composition according to item 10 for use in a method of inhibiting tumour progression in a subject.
14. The antibody according to item 1, the antibody-drug conjugate according to any one of items 2 to 6, or the pharmaceutical composition according to item 10 for use in a method of inhibiting, lowering or eliminating metastatic capacity of an uPARAP expressing tumour.
15. A kit comprising the antibody according to item 1, the antibody-drug conjugate according to any one of items 2 to 6, or the pharmaceutical composition according to item 10, optionally further comprising means for administering the antibody or antibody-drug conjugate to a subject and/or instructions for use.

1. An antibody which binds to uPARAP, the antibody comprising:
a. an immunoglobulin light chain variable region comprising or consisting of the amino acid sequence of SEQ ID NO: 3; and
b. an immunoglobulin heavy chain variable region comprising or consisting of the amino acid sequence of SEQ ID NO: 6.

2. The antibody according to claim 1, wherein the antibody comprises:
a. an immunoglobulin light chain comprising or consisting of the amino acid sequence of SEQ ID NO: 1; and
b. an immunoglobulin heavy chain comprising or consisting of the amino acid sequence of SEQ ID NO: 4.

3. (canceled)

4. An antibody-drug conjugate comprising:
a. the antibody according to claim 1,
b. an active agent, and
c. a linker which links a) to b).

5. The antibody-drug conjugate according to claim 4, wherein the active agent is a therapeutic agent, a radioisotope or a detectable label.

6. (canceled)

7. The antibody-drug conjugate according to claim 4, wherein the active agent is an anti-microtubule agent, an anti-mitotic agent, a DNA crosslinking agent, a DNA alkylating agent, a DNA strand scission agent, an anthracycline, an antimetabolite, a histone deacetylase inhibitor, a kinase inhibitor, a metabolism inhibitor, a peptide antibiotic, an immune checkpoint inhibitor, a platinum-based antineoplastic, a topoisomerase inhibitor, a DNA or RNA polymerase inhibitor, a nucleotide based agent, or a cytotoxic antibiotic.

8. The antibody-drug conjugate according to claim 4, wherein the active agent is monomethyl auristatin E (MMAE), monomethyl auristatin F (MMAF), Paclitaxel, Docetaxel, Vinblastine, Vincristine, Vindesine, Vinorelbine, a mayatansinoid, Colchicine, Podophyllotoxin, cisplatin, carboplatin, oxaliplatin, mitomycin C (MMC), pyrrolobenzodiazepine, dimeric pyrrolobenzodiazepine derivatives, SGD-1882, nitrogen mustards, tris(2-chloroethyl)amine, pyridinobenzodiazepines, indolinobenzodiazepine dimers, Duocarmycin SA, calicheamicin, hamiltrone, Daunorubicin, doxorubicin, epirubicin, idarubicin, PNU-159682, methotrexate, 6-mercaptopurine, 6-thioguanine, fludarabine phosphate, pentostatin, cladribine, 5-fluorouracil, 5-fluorodeoxyuridine, cytarabine, gemcitabine, trichostatin A, vorinostat, belinostat, panabiostat, givinostat, resminostat, abexinostat, quisinostat, rocilinostat, practinostat, CHR-3996, valproic acid, butyric acid, phenylbutyric acid, entinostat, tacedinaline, 4SC202, mocetinostat, romidepsin, nicotinamide, sirtinol, cambinol, EX-527, genistein, lavendustin C, PP1-AG1872, PP2-AG1879, SU6656, CGP77675, PD166285, imatinib, erlotinib, gefitinib, lavendustin A, cetuximab, UCS15A, herbimycin A, radicicol, AP0866, GMX-1777, GMX-1778 ATG-019, and OT-82, Pembrolizumab, Nivolumab, Cemiplimab, JTX-4014, Spartalizumab, Camrelizumab, Sintilimab, Tislelizumab, Toripalimab, Dostarlimab, AMP-224, AMP-514, Atezolizumab, Avelumab, Durvalumab, KN035, CK-301, AUNP12, CA-170, BMS-986189, lipoplatin, nedaplatin, picoplatin, phenanthriplatin, satraplatin, triplatin tetranitrate, camptothecin, topotecan, belotecan, lurtotecan, irinotecan, SN-38, exatecan, Dxd, amanitin, alpha-amanitin, actinomycin D, aphidicolin, or a derivative thereof.

9-19. (canceled)

20. The antibody-drug conjugate according to claim 4, wherein the active agent is a derivative of exatecan.

21. (canceled)

22. The antibody-drug conjugate according to claim 4, wherein the active agent comprises a radioisotope selected from 60Co, 89Sr, 90Y, 99mTc, 131I, 137Cs, 153Sm, and 223Rd.

23. The antibody-drug conjugate according to claim 4, wherein the drug-to-antibody ratio (DAR) is between 1 and 10.

24. The antibody-drug conjugate according to claim 4, wherein the is a cleavable or a non-cleavable linker.

25. The antibody-drug conjugate according to claim 4, wherein the linker comprises or consists of a dipeptide.

26. The antibody-drug conjugate according to claim 4, wherein the antibody-drug conjugate further comprises a spacer.

27. The antibody-drug conjugate according to claim 4, wherein the antibody-drug conjugate further comprises an attachment group.

28-30. (canceled)

31. A polypeptide comprising or consisting of the amino acid sequence of SEQ ID NO: 2 the amino acid sequence of SEQ ID NO: 5.

32. An isolated polynucleotide which encodes the amino acid sequence of any one of SEQ ID NOs: 1, 2, or 3, wherein the polynucleotide further encodes the amino acid sequence of any one of SEQ ID NOs: 4, 5, or 6.

33. (canceled)

34. A vector comprising the polynucleotide according to claim 32.

35. An isolated host cell comprising the polynucleotide according to claim 32.

36. (canceled)

37. A pharmaceutical composition comprising the antibody according to claim 1 and a pharmaceutically acceptable buffer, diluent, carrier, adjuvant, or excipient.

38. A method for treatment of a disease characterised by cells expressing uPARAP, the method comprising administering the antibody according to claim 1 to a subject in need thereof.

39-56. (canceled)

57. A kit comprising the antibody according to claim 1.

58. (canceled)

59. An isolated host cell comprising the vector according to claim 34.

60. A pharmaceutical composition comprising the antibody-drug conjugate according to claim 4 and a pharmaceutically acceptable buffer, diluent, carrier, adjuvant, or excipient.

61. A method for treatment of a disease characterised by cells expressing uPARAP, the method comprising administering the antibody-drug conjugate according to claim 4 to a subject in need thereof.

62. A method for treatment of a disease characterised by cells expressing uPARAP, the method comprising administering the pharmaceutical composition according to claim 37 to a subject in need thereof.

63. A kit comprising the antibody-drug conjugate according to claim 4.

64. A kit comprising the pharmaceutical composition according to claim 37.
